(19) United States
(12) Patent Application Publication
(10) Pub. No.: US 2012/0315322 A1
(43) Pub. Date: Dec. 13, 2012

(54) NANOPARTICLE MEDIATED GENE THERAPY AND THERAPEUTIC PRODUCTS FOR ALZHEIMERS

(75) Inventors: Krishnan Chakravarthy, Williamsville, NY (US); Robert Spengler, Lancaster, NY (US); Tracey Ignatowski, Clarence, NY (US); Siddhartha Venkata Kamisetti, Cleveland, OH (US)

(73) Assignee: NNANOAXIS, LLC, Clarence, NY (US)

(21) Appl. No.: 13/491,785

(22) Filed: Jun. 8, 2012

Related U.S. Application Data
(60) Provisional application No. 61/495,013, filed on Jun. 9, 2011.

(51) Int. Cl. A61K 9/14; A61P 25/28; C07H 21/02; A61K 48/00; A61K 9/127; B82Y 5/00

(52) U.S. Cl. 424/450; 514/44 R; 514/44 A; 424/489; 536/23.1; 536/24.5; 977/754; 977/774; 977/788; 977/907; 977/906

(57) ABSTRACT

The present disclosure provides compositions and methods of treating Alzheimers Disease. In an aspect, a nanoparticle is paired to one or more genetic materials that regulates inflammation in a microenvironment. Such nanoparticles can be used to target predefined target cell types in connection with treatment of at least one of the following Alzheimer's disease, Pick's disease, Lewy Body disease, or Idiopathic dementia.

### CROSS REFERENCED TO RELATED APPLICATION

This application claims the benefit of U.S. Provisional Application No. 61/495,013, filed Jun. 9, 2011, and entitled “Nanoparticle Mediated Gene Therapy for Alzheimer's Disease”, which is incorporated by reference herein in its entirety.

### FIELD

This application relates to the field of therapies for progressive degenerative disease. More specifically, this application concerns the treatment of Alzheimer's disease.

### BACKGROUND

Alzheimer's disease (herein referred to as “AD”) is a progressive and degenerative disease. It is characterized by increased levels of pro-inflammatory cytokines and build-up of toxic β-amyloid depositions, especially in the hippocampus, that gradually destroys memory and the ability to learn. Despite medical advances, there are no definitive therapies for AD. FDA-approved drugs only temporarily slow the worsening of symptoms, and only in about half of the patients who take these medications. This translates into combined direct and indirect costs of AD and other dementias to Medicare, Medicaid, and businesses in excess of $148 billion each year.

Current approved drug treatments for cognitive symptoms of AD include cholinesterase inhibitors, while ‘off-label’ treatment of behavioral symptoms of AD include antidepressant drugs; both of these drug classes, in addition to increasing neurotransmitter availability, elicit unfavorable side effects and inhibit the production of tumor necrosis factor. The problems of high costs, unfavorable side effects, and limited efficacy need to be resolved in treatments of AD. The need exists for therapies that address issues related to AD, such as, high costs, high occurrence of unfavorable side effects, and existing limitations in efficacious treatment of AD.

### SUMMARY

The following presents a simplified summary to provide a basic understanding of some aspects described herein. This summary is not an extensive overview of the disclosed subject matter. It is not intended to identify key or critical elements of the disclosed subject matter, or delineate the scope of the subject disclosure. Its sole purpose is to present some concepts of the disclosed subject matter in a simplified form as a prelude to the more detailed description presented later. In accordance with one or more embodiments and corresponding disclosure, various non-limiting aspects are described in connection with the product for treating AD and methods herein relating to treating AD.

In accordance with a non-limiting embodiment, in an aspect, disclosed is a product, comprising: a nanoparticle paired to W genetic materials that regulates inflammation in a microenvironment and X. predefined targeting moieties that correspond to Y predefined target parameters associated with Z predefined target cell types in connection with treatment of at least one of the following Alzheimer's disease, Pick's disease, Lewy Body disease, or Idiopathic dementia, wherein W, X, Y, and Z are integers.

In various aspects, the product comprises genetic materials that is L tumor necrosis factor inhibiting small inhibitory RNA (siRNA) sequences that respectively silences M genes that produce a tumor necrosis factor (TNF), wherein L and M are integers. In another aspect, the product comprises genetic materials that are N plasmids that enables the predefined target cell types to upregulate at least one of IL-4, IL-10; or IL-13, wherein N is an integer.

Further, according to another non-limiting embodiment, the product decreases or prevents an increase of at least one or more of β-amyloid formation, and neurofibrillary tangle formation by facilitating functioning of tau protein in brain cells. In another non-limiting embodiment, the nanoparticle is any one or more of a biodegradable polymer, tetrapod quantum dot, tetrapod article, multi-legged luminescent nanoparticle, tetrapod nanocrystal, biodegradable nanoparticle, liposome, nanocarrier, or dendrimer. In yet another embodiment, the nanoparticle is further paired to at least one or more of: TNF antagonist selected from the group consisting of etanercept soluble TNF receptor Type I, pegylated soluble TNF receptor TYPE I (PEGS TNF-R1), or onercept. In yet another aspect, the nanoparticle is further paired to a biological substance that at least causes one or more of increased IL-10 production, increased IL-4 production, decreased IL-1 production, decreased IL-6 production, or increased IL-13 production.

The disclosure further provides a method, comprising administering to a subject a product comprising a nanoparticle bound to at least one or more TNF inhibiting siRNA sequence or genetic material that silences one or more genes that encode for TNF in connection with treatment of at least one of the following diseases Alzheimer's disease, Pick's disease, Lewy Body disease, or Idiopathic dementia.

Other embodiments and various non-limiting examples, scenarios and implementations are described in more detail below. The following description and the drawings set forth certain illustrative aspects of the specification. These aspects are indicative, however, of but a few of the various ways in which the principles of the specification may be employed. Other advantages and novel features of the specification will become apparent from the following detailed description of the specification when considered in conjunction with the drawings.

### BRIEF DESCRIPTION OF THE DRAWINGS

FIG. 1 illustrates an example non-limiting pathophysiology of AD in the brain and pathophysiology effects from the nanoparticle gene mediated product for treatment of AD, Pick's disease, Lewy Body disease, or Idiopathic dementia,

FIG. 2 is an exemplary non-limiting block diagram representing an exemplary non-limiting of product 200 for treatment of AD.

FIG. 3 illustrates an example non-limiting chart that displays a nanoparticle paired to genetic material to demonstrate the knockdown of gene expression in a microenvironment.

FIG. 4 is an exemplary non-limiting block diagram of a nanoparticle paired to more than one genetic materials.

FIG. 5. illustrates example non-limiting images that demonstrate a nanoparticle paired to genetic materials in a microenvironment that is the CA1 region of a rat hippocampus.

FIG. 6 illustrates example non-limiting images of quantified immunoreactive staining for TNF in the rat hippocampus.

FIG. 7 is an exemplary non-limiting block diagram of a nanoparticle that can pair to genetic materials that are plasmids.

FIG. 8 illustrates example non-limiting that determine the cell type(s) in which the uptake of a nanoparticle paired to a plasmid was associated in vivo, rat coronal hippocampal sections.

FIG. 9 is a bar graph that illustrates the induction of gene expression of TNF 21 days, after bilateral GNR-pTagrfp-Mutnf nanoplasmidex microinjection into the rat hippocampal CA1 region.

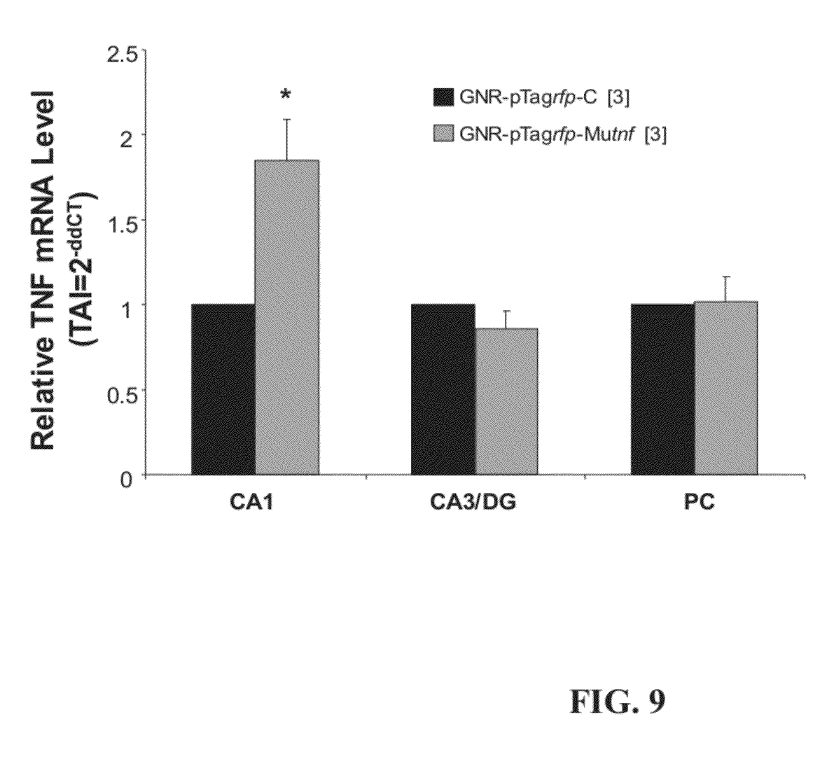

### DETAILED DESCRIPTION OF SEVERAL EMBODIMENTS

### Overview

The innovation is now described with reference to the drawings, wherein like reference numerals are used to refer to like elements throughout. In the following description, for purposes of explanation, numerous specific details are set forth in order to provide a thorough understanding of this innovation. It may be evident, however, that the innovation can be practiced without these specific details. In other instances, well-known structures and components are shown in block diagram form in order to facilitate describing the innovation.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.” In case of conflict, the present specification, including explanations of terms, will control. In addition, all the materials, methods, and examples are illustrative and not intended to be limiting.

By way of introduction, the subject matter disclosed in this disclosure relates to novel products, therapies and methods of that are effective for treating Alzheimer's disease. Tumor necrosis factor (hereinafter referred to as “TNF”) is a pleotropic immune signaling molecule and also a pivotal regulator of neuron synaptic function and growth function. Increased levels of TNF are involved in the pathogenesis of a variety of inflammatory neurological disorders, including, but not limited to, Alzheimer's disease. Increased levels of tumor necrosis factor-alpha (TNF-α), a proximal pro-inflammatory cytokine, in the brain induces neuroplastic changes associated with several neural disorders, including Alzheimers Disease. Many cell types, including neurons, produce TNF in the brain. As a neuromodulator, TNF regulates synaptic function in the brain; therefore, it follows that its' over-expression mediates the synaptic dysfunction evident in AD.

Thus relief from symptoms of AD is linked to inhibition of TNF production. Reduction of TNF production in the hippocampus prevents amyloid betal-42 protein (Aβ1-42)-induced neurotoxicity and neurofibrillary tangle (NfT) formation. The product uses a combination of nanotechnology and small inhibitory RNA (siRNA) to selectively inhibit TNF production in cultured hippocampal cells exposed to Aβ1-42. Reduction of TNF production prevents neurotoxicity and formation of NfTs in neuron cell cultures.

This product treats Alzheimer's Disease by applying siRNA mediated gene silencing in the central nervous system (CNS) to target the gene encoding for TNF, a neuromodulator of the serotonergic, noradrenergic, cholinergic, and dopaminergic pathways that play a central role in Alzheimer's disease. RNA silencing achieves significant and persistent TNF gene knockdown to alter neuronal plasticity. Nanoparticles enhance the therapeutic application of siRNA to prevent siRNA rapid degradation, enhance cellular uptake, allow for controlled release of genetic materials, as well as target cells. In one embedment, the methods, therapies and products comprise a formulation of polymer nanoparticle, such as poly-lactide-co-glycolide polyethylene glycol (PLGA-PEG) that is both highly biocompatible for conjugation to siRNA and biodegradable for sustained release of siRNA.

### Role of TNF in Brain in the Pathophysiology and Treatment of Alzheimer's Disease

Alzheimer's disease (AD) is a disease wherein early on in the disease process, localized pathological changes induced by TNF can be noticed, such as, (β-amyloid plaque deposition, neurofibrillary tangle (NfT) formation, and atrophy, which are all evident in the hippocampus of a subject. Localized targeting in the hippocampus region to inhibit TNF supply to hippocampal neurons provide relief from the ravages of AD. For instance, β-amyloid stimulates TNF production in the brain that contributes to the ongoing inflammatory cascade, snaptic dysfunction, and neuron death characteristic of AD.

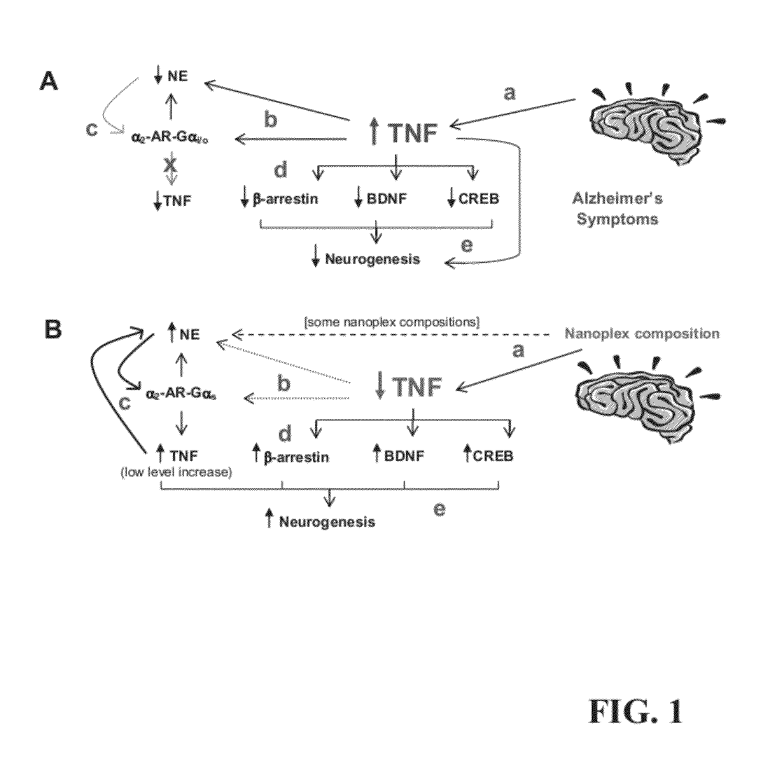

Referring now to the drawings, with reference initially to FIG. 1, illustrated is an example pathophysiology of AD in the brain and pathophysiology effects from the nanoparticle gene mediated product for treatment of AD. The pathophysiology in FIG. 1(A) demonstrates the role of TNF in induction of symptoms and behavior of AD subjects. As demonstrated in (a), AD causes a proinflammatory cytokine cascade, wherein TNF is the proximal cytokine As shown in (b), TNF inhibits stimulated norepinepherine (NE) release, and TNF enhances Gαᵢ protein expression. The presynaptic α₂-adrenergic receptor that normally favors coupling to Gαᵢ and inhibition of NE release becomes supersensitized (e.g. greater coupling to Gαᵢ protein) with greater inhibition of NE release. At (c), the deficiency in bioavailable NE results in attenuated α₂-adrenergic receptor (α₂-AR) stimulation that upon activation normally inhibits TNF expression; therefore, no compensatory decrease in TNF occurs, producing a sustained elevation in TNF levels. Shown in (d), the lack of inhibited TNF expression causes a decrease in brain-derived neurotrophic factor (BDNF), CRE-binding protein (CREB), and β-arrestin activity. Furthermore, the decrease in β-arrestin activity prevents desensitization of the G-protein-coupled receptor (α₂-AR-Gα_{i/o}) Thus, although normally low levels of TNF stimulate BDNF production, elevated levels of TNF inhibit BDNF production Similarly, TNF produces a decrease in CREB. As shown in (e), each of, elevated TNF levels, decreases in β-arrestin, decreases in BDNF, and decrease in CREB, alone or in various combinations, are all causes of decreased neurogenesis that is linked to AD.

Turning now to FIG. 1(B), the role of TNF in the mechanism resulting from administration of the nanoparticle mediated gene therapeutic product is shown. As demonstrated in (a), initially, upon dosing, the product 200 (as later disclosed) decreases TNF levels through a blockade of a gene that, in turn, increases NE availability allowing for decreased α₂₋AR activation (it is coupled to Gα_{i/o} protein); the α₂₋AR-Gα_{i/o} configuration supports a decrease in TNF (see ‘c’ above in (A) of FIG. 1). This initial decrease in TNF expression is a cAMP-mediated event As shown in (b), as TNF normally inhibits NE release, the initial decrease in TNF levels allows for disinhibition of (increase in) NE release. Also, TNF produces an increase in Gα_{i/o} protein expression so decreased TNF results with a decrease Gα_{i/o} protein expression.

This decrease in Gα_{i/o} protein expression, caused by product 200, allows for α₂₋AR coupling to Gαₛ proteins that upon activation now facilitates NE release. In (c), it is shown that the increase in bioavailable NE allows for enhanced α₂₋AR activation that not only serves to further increase NE release, but also now supports an increase in TNF production. In turn, TNF now increases NE release (directly). A new baseline is thus established between TNF production and α₂₋AR coupling for balanced regulation of NE release. As shown in (d), the overall decrease in TNF (from pathological to physiological levels) allows for increased β-arrestin activity, and BDNF and CREB production. It is demonstrated in (e) that enhanced BDNF, CREB, β-arrestin activity, increased monoamine levels, and lowered TNF levels all support neurogenesis, and a remedy to AD in that there is a decrease or less β-amyloid plaque deposition, decreased or less neurofibrillary tangle (NfT) formation, and decreased or less atrophy in a subject.

### Example Embodiments of Nanoparticle Mediated Gene Therapy and Therapeutic Products for Alzheimers Disease

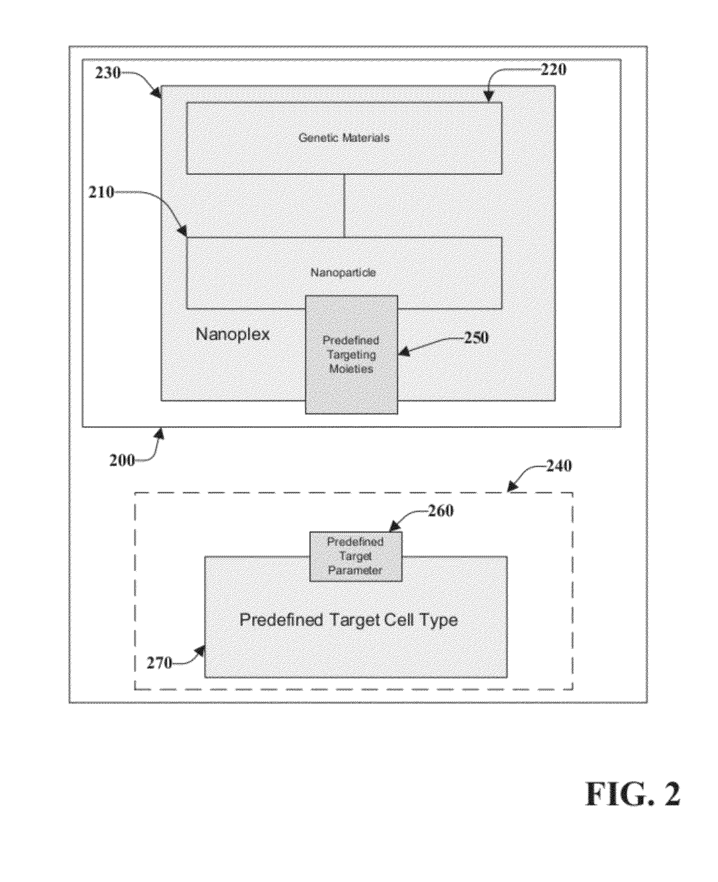

Turning now to FIG. 2, presented is an exemplary non-limiting embodiment of product 200 for treatment of a disease. In an aspect, product 200, administered to a subject, will treat any one or more of: Alzheimer's disease, Pick's disease, Lewy Body disease, or Idiopathic dementia. In an embodiment, product 200, comprises a nanoparticle 210 paired to W genetic materials 220 that regulates inflammation in a micro-environment 240 and nanoparticle 210 paired to a X predefined targeting moieties 250 that correspond to Y predefined target parameters 260 associated with Z predefined target cell types 270 in connection with treatment of at least one of the following Alzheimer's disease, Pick's disease, Lewy Body disease, or Idiopathic dementia, wherein W, X, Y, and Z are integers.

In an embodiment, product 200 employs a nanoparticle 210, a genetic materials 220, and a predefined targeting moieties 250. In an aspect, the nanoparticle 210 is paired to W genetic materials that regulates inflammation in a micro-environment and is paired to predefined targeting moieties 250. Nanoparticle 210 is a particle ranging in size from 0.001 nm to 999.999 nm, of any shape, any size distribution, any form, and any material compositions. In an aspect, In an aspect, nanoparticle 210 is any one or more of a nanoparticulate, nanoparticle, nanocrystal, molecule or particle that is biodegradable, biocompatible, or non-biodegradable. In an embodiment, nanoparticle 210 can encapsulate therapeutic agents, like low molecular weight drugs, macromolecule's such as a protein, small inhibitory RNA (siRNA), or plasmid DNA. In another embodiment, nanoparticle 210 control releases a drug or genetic material or both a drug and genetic material in a sustained manner. In an aspect, the properties of nanoparticle 210 allow for targeted drug delivery or targeted delivery of genetic material, which can include, but is not limited to, cellular delivery or delivery to organelle targets. Nanoparticle 210, in an aspect, has the ability to exploit biological pathways, such as the pathophysiology of TNF in the brain, accomplish the delivery of a drug payload to cellular or intracellular targets, and transport genetic materials 220 and/or targeting moieties past the blood-brain barrier.

In various embodiments, nanoparticle 210 is any nanoparticle type, shape, material composition, size distribution, and form. For instance, in an aspect nanoparticle 210 is any one or more of an inorganic nanoparticle, polymeric nanoparticle, solid nanoparticle, lipid nanoparticle, liposome, nanocrystal, nanotube, dendrimer, biodegradable dendrimer, polymer-based dendrimer, branched nanoparticle, gold nanoparticle, silver nanoparticle, iron nanoparticle, carbon nanotube, layered double hydroxide nanoparticle, silica nanoparticle, iron oxide nanoparticle, calcium phosphate nanoparticle, fullerene, or quantum dot. In other aspects nanoparticle 210, is any one or more nanosphere, nanocapsule, hydrophobic polymer, polymeric nanoparticle's, luminescent tetrapod dots, multi-leg luminescent nanoparticle, iron nanoparticle, phosphorous quantum dot, cadmium free quantum dot, semiconductor nanocrystal, polymer, ceramic nanoparticle, nanopolymer made from chitosan.

In some embodiments, nanoparticle 210 is comprised of various material compositions or forms. For instance, in an aspect, nanoparticle 210 is comprised of a core and layer or shell structure, wherein the core of the nanoparticle is comprised of one material and F layers are comprised of either the same or another material, wherein F is an integer. For example, if F equals two, nanoparticle 210 can be a nanocrystal comprised of a CdSe core material, a CdS layer, and a silica (glass) coating layer. Thus there are two layers coating the nanocrystal core in this example embodiment of nanoparticle 210. The layers can be of various coat materials such as glass, silica, polymer, amphiphilic coating, neutral polyethylene glycol (PEG), cationic (PEG-amine), anionic coating, carboxylic acid, . . . ) Furthermore, several functional groups can be prepared on the outer layer of an inorganic nanoparticle, the functional layer can be any number of functional groups. For instance a functional layer can be a saturated hydrocarbon, unsaturated hydrocarbon, carboxylic acid, thiol, amine, or alcohol.

In an embodiment, nanoparticle 210 is a polymeric nanoparticle that is biodegradable. In another aspect, the polymeric nanoparticle is synthesized from any one or more of gelatin, chitosan, poly(lactic-co-glycolic acid) copolymer, polylactic acid, polyglycolic acid, or poly(alkylcyanoacrylate. In another embodiment, nanoparticle 210 is a biodegradable polymer that degrades over time when introduced into the body of a subject. The biodegradable polymer may be composed of any one or more of polyesters, polycarbonates, polyketals, PLGA or polyam ides. Such polymers may comprise polycaprolactone, block-co-polymer of a polyether, such as poly(ethylene glycol), and a polyester, polycarbonate, polyamide, block-co-polymer of poly(ethylene glycol) and poly(lactic acid), poly(glycolic acid), poly(lactic acid-co-glycolic acid), PLGA, poly(lactide-co-glycolide), or polycaprolactone.

In another embodiment, nanoparticle 210 is a polymeric nanoparticle with an outer layer coating such as a poly-ethylene-glycol (PEG) coating and possess a biodegradation profile to facilitate biocompatibility in a subject. In another embodiment, nanoparticle 210 can be a lipid-based colloidal nanoparticle comprising a core hydrophobic lipid material that is epitaxially covered by a monolayer of phospholipids. In other aspects, nanoparticle 210 can take any one of a variety of material compositions with nano-dimensions. In some embodiments, nanoparticle 210 can be polymers that are linear or branched polymers. In some embodiments, polymers can be dendrimers. In some embodiments, polymers can be substantially cross-linked to one another. In other embodiments, polymers can be substantially free of cross-links. In another aspect, polymers can be used in accordance with the present invention without undergoing a cross-linking step. It is further to be understood that inventive compounds and synthetic nanocarriers may comprise block copolymers, graft copolymers, blends, mixtures, and/or adducts of any of the foregoing and other polymers. The polymers listed herein represent an exemplary, not comprehensive, list of polymers that can be of use in accordance with the present invention.

In another aspect, nanoparticle 210 is paired to genetic materials 220 that regulates inflammation in a microenvironment. Genetic materials 220 means the form of, for example, a live gene, DNA, RNA, siRNA, or an oligonucleotide duplex. The nanoparticle 210 is paired to one or more genetic materials 220 wherein paired is meant to describe the adherence between the nanoparticle 210 and the genetic materials 220, either directly or through a functional moiety capable of adhering nanoparticle 210 to one or more genetic materials 220. In an aspect, the pairing can comprise any sort of magnetic interactions, electrostatic charge interactions, affinity interactions, charge interactions, metal coordination, physical adsorption, dipole-dipole interactions, hydrophobic interactions, stacking interactions, bond, including, but not limited to, covalent, non-covalent, ionic, hydrogen bonding, Van der Waals forces, mechanical bonding. In another aspect, the nanoparticle 210 can be a nanocarrier, wherein the pairing to genetic materials 220 is via containment of genetic materials 220 within the nanocarrier, wherein the nanocarrier encloses genetic materials 220 such that it is not exposed to an environment until delivery of genetic materials 220 to predefined target cell types 270. In an aspect, pairing that utilizes functional groups can include linkers, polymers, or linking agents.

Accordingly, in some embodiments, linking agents, are used to pair nanoparticle 210 to genetic materials 220, such linking agent can be a polyester, poly(ethylene glycol), poly(lactic acid), poly(glycolic acid), poly(lactic-co-glycolic acid), or a polycaprolactone. Furthermore, in an embodiment, the linking agent can be any one or more of N-(3-aminopropyl)3-mercapto-benzamide, 3-aminopropyl-trimethoxysilane, 3-mercaptopropyl-trimethoxysilane, 3-maleimidopropyl-trimethoxysilane, 3-hydrazidopropyl-trimethoxysilane, succinimidyl esters, or maleimides, iodoacetamides. In another aspect, the linking agent can be a moiety that links nanoparticle 210 to genetic materials 220. In an aspect, the moiety acts as a biological bridge that can include but is not limited to, chemical chains, chemical compounds, carbohydrate chains, peptides, or haptens. bifunctional reagents/linker molecules, biotin, acidin, free chemical groups (e.g. thiol, carboxyl, hydroxyl, amino, amine, sulfo, etc.), or reactive chemical groups.

Pairing can occur by a linking agent, for example, nanoparticle 210 can be a quantum dot and genetic materials 220 is a polynucleotide wherein a carboxyl group on the surface of the quantum dot forms a bond with the hydroxyl group of the polynucleotide. The linkages via a linking agent can be cleavable in some aspects such as with sulfosuccinimidyl-2-(p-azido salicylamido) ethyl-1,3′-dilithiopropionate. In an aspect, the linker can be diaminocarboxylic acid such as lisine, asparagine, glutamine, arginine, citrulline, ornithine, 5-hydroxylisine, djenkolic acid, β-cyanoalanine, 3,5-diaminobenzoic acid, 2,3 diaminopropionic acid, 2,4-diaminobutyric acid, 2,5-diaminopentanoic acid, 2,6-diaminopimelic acid. In other embodiments, the linking agent can be an amine group, carboxyl group, hydroxyl group, sulfhydryl group, monoaminocarboxylic acid group. In an aspect, the linking agent can be any chemical modification of the surface of nanoparticle 210 that enables pairing to genetic materials 220.

In another aspect, product 200 comprising of nanoparticle 210 paired to genetic materials 220 may also comprise predefined targeting moieties 250 on the surface or within nanoparticle 210. Predefined targeting moieties 250 means a target feature, such as an affinity molecule paired to nanoparticle 210 that has an affinity for sites of action in cells, such as antigen presenting cells (APC's). For instance, the predefined targeting moieties 250 can be any one or more of monoclonal antibody, polyclonal antibody, nucleic acid (monomeric or oligomeric,), protein polysaccharide, small molecules, sugar, peptide, drugs, ligands, or any other such affinity molecule. The genetic materials 220 can dissociate from the nanoparticle 210 (e.g. dissociation resulting from environmental pH changes) upon association with a predefined target cell type 270.

In an aspect, predefined target cell type 270 can be any cell type associated with AD. For instance predefined target cell type 270 can be any one or more of various nerve cells in the brain, such as neurons, glial cells (e.g. astrocytes, microglia, etc.), accessory cells or endothelial cells. In some aspects, predefined targeting moieties 250 possess an affinity for predefined target parameter 260. In an aspect, predefined targeting moieties 250 may pair with predefined target parameter 260 in a microenvironment 240. In an aspect, predefined target parameter 260 may be a detectable substance associated with predefined target cell type 270, wherein the presence or absence of the detectable substance ascertains the identification of predefined target cell type 270, wherein nanoparticle 210 can further deliver genetic materials 220 to such predefined target cell type 270. In an aspect, predefined target parameter 260 may be an intracellular receptor, extracellular receptor, antigen, or protein, both in the cell as well as on the cell surface. The outcome of AD depends on mechanisms that are unique to an organ microenvironment. For instance, the local microenvironment 240 may cause selective, local neurite degeneration by loss of apoptotic-like mechanisms, leading to the loss of synaptice connectivity observed in AD brain. Accordingly, in another aspect, microenvironment 240 means a site of disease in the brain that is specialized and effectively isolated (e.g. nerve cell). Thus, microenvironment 240 can be, but is not limited to, endothelial cells, cells of myeloid origin, astrocytes, glial cells, microglial cells, neurons.

In another aspect, nanoparticle 210 is efficacious in delivering genetic materials 220 to regions in the brain because of the nano-scale size and/or shape of the product 200, which may penetrate between prominent tight junctions between brain endothelial cells and metabolic barriers that prevent the passage of other small molecules and cells through the blood-brain barrier (BBB). Nanoparticles 210 can be paired to transferrin to act as a targeting ligand, thereby allowing product 200 to cross the BBB due in part to the expression of transferrin receptors by the endothelial cells that line the BBB. Product 200 can pass through the blood-brain barrier (BBB), which is a tight-knot layer of endothelial cells that coats miles of capillaries and blood vessels in the brain. While the BBB sequesters the brain from potential harm, it also obstructs delivery of neurological drugs to a site of disease in the brain of a subject. A subject means mammals and non-mammals, not denoting a particular age or sex, including, but not limited to, humans, chimpanzees, other apes and monkey species, farm animals (e.g. swine, cattle, horses, sheep, goats), domestic animals (e.g. rabbits, dogs, cats, . . . ), laboratory animals (e.g. rodents, rats, mice, guinea pigs, etc.), or birds. In some aspects, nanoparticle 200 can pair to more than one predefined targeting moieties 250. For example nanoparticle 200 can pair to one targeting moieties 250 that carries product 200 across the blood brain barrier and a nanoparticle 200 can pair to a second targeting moieties 250 that carries product 200 to a predefined target cell type 270 located in microenvironment 240.

In an embodiment, product 200, is comprised of a nanoparticle 210 paired to W genetic materials 220 that regulates inflammation in a microenvironment 240 and X predefined targeting moieties 250 that correspond to Y predefined target parameters 260 associated with Z predefined target cell types 270 in connection with treatment of at least one of the following Alzheimer's disease, Pick's disease, Lewy Body disease, or Idiopathic dementia, wherein W, X, Y, and Z are integers. In an aspect, nanoparticle 210 can be paired to W genetic materials 220, wherein W is an integer. In an embodiment, one or more genetic materials 220 decrease TNF levels through blockade of TNF gene expression. In an aspect, the inhibition of TNF is limited to the central nervous system and the levels of TNF decreased are only reduced to physiological levels of TNF that are necessary for proper functioning.

In an aspect, nanoparticle 210 is paired to X predefined targeting moieties 250, wherein X is an integer. For instance, if predefined targeting moieties 250 is an antibody and X equals three, then nanoparticle 210 is paired to three of the antibody. Furthermore, if predefined targeting moieties are two different antibodies and X=3, then nanoparticle 210 can be paired to two of the same antibodies and one of different antibody. Each predefined targeting moieties 250 can allow product 200 to have an affinity for Y predefined target parameters 260, wherein Y is an integer. For instance, if predefined target parameters 260 are extracellular receptor sites on predefined target cell type 270 and Y equals four, then product 200 can have an affinity for any of the four different extracellular receptor sites respectively. In an aspect, product 200 can target Z predefined target 270, wherein Z is an integer. For instance, if Z equals two, then product 200 can target two different predefined target cell type 270. Thus if the predefined target cell types 270 are astrocytes and microglial cells, then product 200 can target microglial cells and product 200 can target astrocytes as well. For example, nanoparticle 210 can be a biodegradable polymer, such as, poly-lactide-co-glycolide polyethylene glycol (PLGA-PEG), wherein the PLGA-PEG is paired to three siRNA sequences coded for TNF gene knockdown and which regulate inflammation in microenvironment 240, the hippocampus region of the brain. Furthermore, nanoparticle 210 can also be paired to predefined target moieties 250, such as a ligand and antibody to target the nanoplex 230 to transfer across the blood brain barrier (BBB) and deliver the siRNA payload (or other genetic materials 220) to predefined target cell types 270, such as glial cells, by attracting to predefined target parameters 260, such as extracellular surface proteins, intracellular proteins, or receptor regions present on or within predefined target cell type 270.

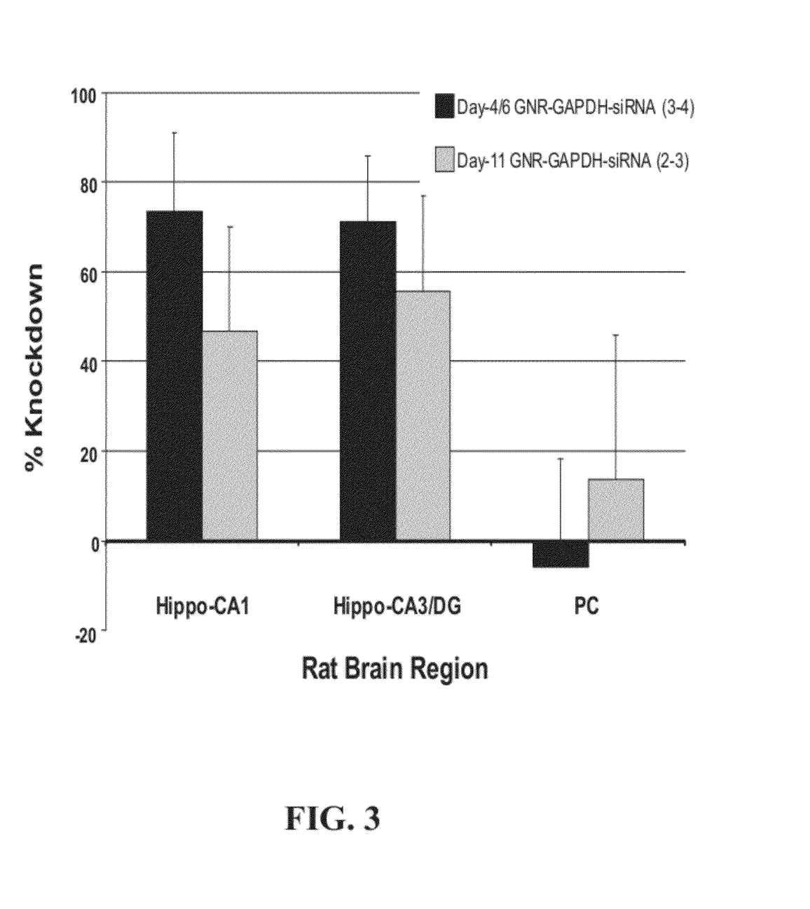

Turning now to FIG. 3, illustrated is a chart that displays a nanoparticle paired to a genetic materials to demonstrate the knockdown of gene expression in a microenvironment. In an aspect, a nanoparticle 210 that is a gold nanorod (GNR) is paired to genetic materials 220 that is GADPH siRNA-Cy3 which targets microenvironment 240, a rat hippocampus. FIG. 3 demonstrates knockdown of GAPDH gene expression using GNR-GAPDH siRNA-Cy3 in the rat hippocampus. A single injection (6 μl; 0.5 μl/min) of GNR-GAPDH siRNA-Cy3 (500 ng/1 nmol) was administered into the CA1 region of the right hippocampus or GNR-scrambled siRNA-Cy3 in the left hippocampus. The Q-RT-PCR data shows >70% suppression of GAPDH gene expression in the CA1 region and in the combined CA3/dentate gyrus regions of the hippocampus at 4 days post-injection. This level of suppression was maintained for up to 11 days post-injection. The brain region overlying the injection site, part of the parietal cortex, was used as a control region for diffusion. Results are expressed as the mean ±SEM with the n number indicated in parentheses. As published, binding of siRNA with GNRs and loading efficiency was monitored by agarose gel electrophoresis mobility [24]. Hippo, hippocampus; DG, dentate gyrus; PC, parietal cortex. Gene knockdown was accomplished as per the configuration of nanoparticle 210 paired to genetic materials 220 demonstrated in FIG. 3, however, other more suitable nanoparticle 210 exist and genetic materials 220 that are TNF inhibiting siRNA may be used.

In another embodiment, the nanoparticle 210 is a hydrophobic biodegradable polymer PLGA that is dissolved in organic solvent (chloroform) to make a stock solution (10 mg/ml). PLGA stock solution (0.5 ml) and 2.5 ml of DSPE-PEG solution (20 mg/ml in chloroform) are mixed (1:10), and organic dye (100 μl, 5 mg/ml in chloroform) is added to the solution, followed by evaporating the solvent. The residue is dispersed in 10 ml of HPLC water by gently stirring, after which, the dispersion is filtered by a 0.2 μm filter. For polymer coating, 250 μl of 1:20 diluted PDDAC aqueous solution is added to 5 ml of the particle dispersion under gentle stirring. The following are used for preparing PLGA-PEG nanoparticles: Poly(DL-lactide-co-glycolide) (PLGA, average Mw 5,000-15,000, lactide:glycolide (50:50)) (Sigma-Aldrich), Poly(diallyldimethylammonium chloride) (PDDAC, 20%) (Polysciences, Inc.), 1,2-Distearoyl-sn-Glycero-3-Phosphoethanolamine-(Polyethylene Glycol)2000 (DSPE-PEG) (Laysan Bio Inc.), and Organic dye LD688 (Exciton, Ohio). Silencer® Pre-designed TNF siRNA-Cy□-3 labeled (sense: 5′- GCAGAUGGGCUGUACCUUAtt-3′, antisense: 5′-UAAGGUACAGCCCAUCUGCtg-3′) or Silencer® Control #1 siRNA-Cy□-3 labeled (validated, non-targeting siRNA used as a negative control; referred to as scrambled siRNA) (Ambion Inc., Austin, Tex.) can be used.

The siRNAs can be paired to biodegradable polymer PLGA (nanoparticle 210 in this aspect) by electrostatically pairing the siRNA with the cationic polymer nanoparticles by incubating 9 pmoles of siRNA and 100 μl of nanoparticles in PBS solution for 4 hr at 37° C. The sample is filter sterilized prior to transfection/injection. Dynamic light scattering (DLS) and zeta-potential measurements are used to estimate the hydrodynamic diameter and surface charge of the nanoparticles and nanoplexes, respectively, using a Brookhaven 90 Plus ZetaPALS Dynamic Light Scattering Instrument. In other embodiments, the nanoparticle 210 is not a biodegradable PLGA polymer.

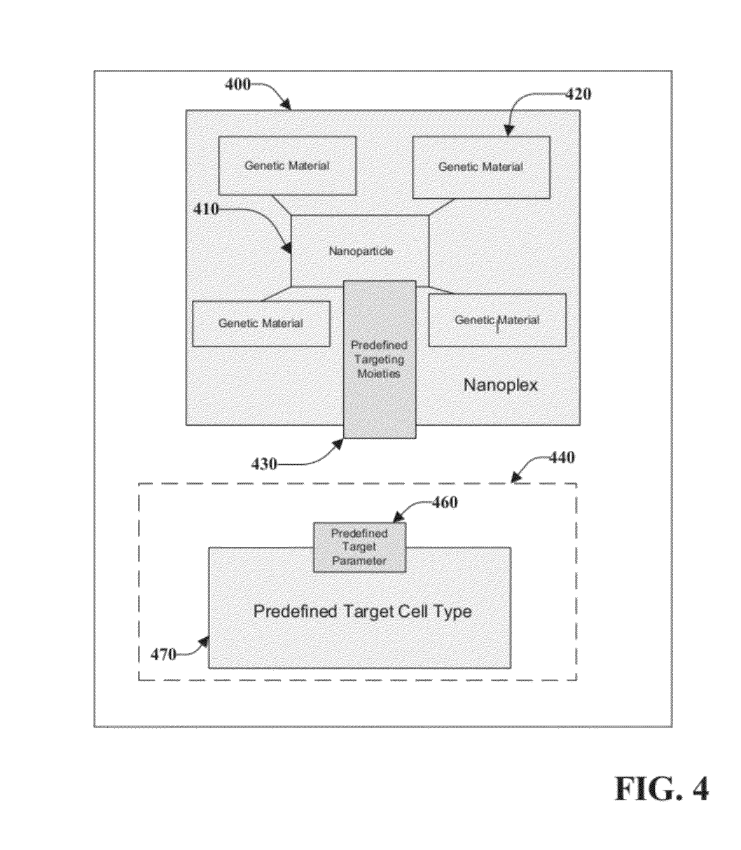

Turning now to FIG. 4, product 400, comprises a nanoparticle 210 paired to more than one genetic materials 420, wherein the genetic materials 420 in an aspect is L tumor necrosis factor inhibiting small inhibitory RNA (siRNA) sequences that respectively silences M genes that express a tumor necrosis factor (TNF), wherein L and M are integers. For instance, if L equals three, nanoparticle 210 can be paired to three different siRNA nucleotide sequences, each of which inhibits TNF. Accordingly, if L equals three, nanoparticle 210 can be paired to three of the same siRNA nucleotide sequence, each of which inhibits expression of TNF. Furthermore, if L equals three, nanoparticle 210 can be paired to two of the same siRNA nucleotide sequences and one different siRNA nucleotide sequence, each of which respectively inhibits expression of TNF. Additionally, if M equals three, product 400 can silence three different gene sequences, each of which respectively, if expressed, cause TNF to be produced.

In an aspect, genetic materials 420 can be any one or more of a multitude of tumor necrosis factor (TNF) inhibiting small inhibitory RNA. The nanoparticle 410 can be used for both in vitro and in vivo delivery of TNF inhibiting siRNA, wherein upon delivery of the siRNA to a predefined target cell type 470, gene expression of TNF is blocked, in some embodiments, the blocking occurs in the microenvironment 440 that is the hippocampus region of the brain. TNF inhibition can occur as a result of the siRNA control of mRNA translation and degradation. An siRNA is able to specifically interact with a respective target messenger RNA (mRNA) that control the regulation of gene expression, and lead to discrete downstream consequences, such as a decrease in TNF production by interfering with mRNA translation. In an aspect, genetic materials 420 can be any one or more siRNA sequences and variations for all animals including human, rodent, primates and other such subjects. Each siRNA is comprised of a nucleotide sequence that is complementary to a portion of the sequence of one or more target messenger RNA (mRNA) to enable gene silencing. Gene silencing is a process by which the expression of a specific gene (e.g. gene expressing production of TNF) is lessened or attenuated. The gene silencing decreases the expression of a gene product (e.g. TNF) that results from siRNA, which sometimes act in concert with host proteins to degrade mRNA.

In an embodiment, the TNF that is inhibited by siRNA genetic materials is the proximal pleiotropic cytokine tumor necrosis factor-alpha (TNF-α) which is involved in the pathogenesis of AD. TNF-α is essential to the cognitive experience of AD and associated symptoms related to AD, such as memory loss, neurofibrillary tangle, or β-plaque formation. For instance, inhibition of TNF activity prevents intraneuronal amyloid precursor protein accumulation. Hippocampal noradrenergic neurotransmission is dampened following elevated levels of hippocampus-TNF. TNF-regulated release of brain norepinephrine is dependent upon α₂-adrenergic receptor activation, and the presynaptic α₂-adrenergic receptor is a principle regulator of norepinephrine release. There is a correlation between α₂-adrenergic receptor inhibition of norepinephrine release and upregulated levels of brain-TNF during AD. The sustained elevated levels of TNF limited to the hippocampus induce AD behaviors. Consequently, product 400 and other embodiments herein can be designed to apply gene transfection to inhibit TNF production specifically in the hippocampus. In some embodiments, product 400 can also inhibit downstream signaling molecules to TNF as described later in the specification.

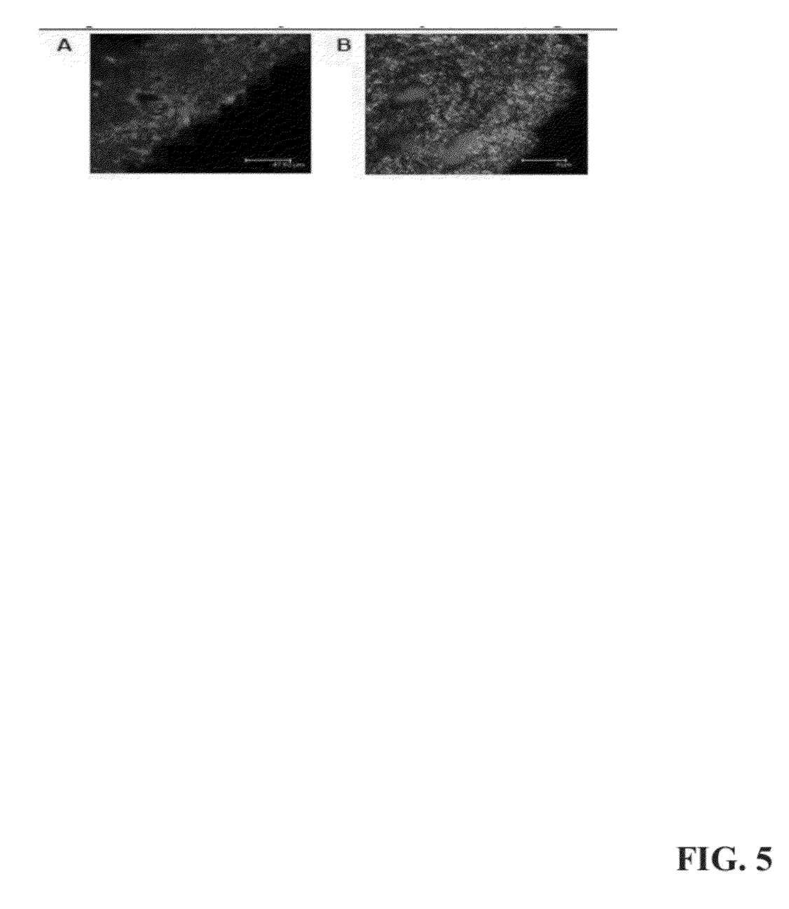

Turning now to FIG. 5, the images demonstrate a nanoparticle paired to genetic materials in a microenvironment that is the CA1 region of a rat hippocampus. The hippocampus is part of the limbic system of the brain, which is comprised of multiple subregions that together are referred to as the hippocampal formation, the subregions include, but are not limited to; Ammon's horn or the hippocampus proper (CA1, CA2, and CA3) and the dentate gyrus, which are all interconnected. The hippocampal formation (hippocampus) is one of the first structures affected by NfT formation in AD; it is also the most severely affected brain region in advanced stages of AD. The hippocampus is well known for its involvement in long-term potentiation associated with learning and memory; thus, the pathological changes in this region account for the memory impairment that is a prominent feature of AD. Such changes include β-amyloid plaque formation, Nft formation, and marked cell loss74,75, resulting in hippocampal atrophy.72,73 ICV injection of Aβ1-42 protein into the rodent brain, a method to induce AD pathology, increases TNF expression, especially in the hippocampus. The results focus attention on glial production and neuronal production of TNF. Initially, as shown in FIG. 5, GNR-siRNA nanoplexes were used to silence gene expression in the hippocampus. In some aspects, a nanoparticle that is bioconjugated to an siRNA can be referred to as a nanoplex.

Demonstrated in FIG. 5 is cellular uptake of GNR-conjugated glyceraldehyde 3-phosphate dehydrogenase (GAPDH)-siRNACy3 in rat hippocampus neuronal cells using microinjection into the CA1 region.78 Accomplished was a 70% knockdown of GAPDH in the CA1 region, 11 days after a single microinjection. Nanoplexes co-localized with neurons in the CA1 region of the hippocampus thereby showing that the siRNA nanoplexes can effectively and efficiently knockdown gene expression in select regions of the rat brain. Furthermore, as demonstrated in FIG. 5 is multi-labeling immunofluorescent staining of rat coronal hippocampal sections (10 μm, acetone-fixed). The nanoplex GNR-GAPDH-siRNA was microinjected into the CA1 region of the hippocampus and isolated 24 hours later. Turning now to FIG. 5(A) is an image that demonstrates co-localization of GNR-GAPDH-siRNA staining (green), nuclear (Hoechst dye, 10 μM) staining (blue), and glial cells (red). Turning now to FIG. 5(B) is a visualization of GNR-GAPDH-siRNA staining (green), nuclear (Hoescht dye) staining (blue), and Neurofilament-200, NF-200 (1:30,000 Sigma) staining for neurons (red), and goat anti-mouse IgG1 -AlexaFluor 647 (1:2,000 Invitrogen) secondary antibody (red) was used for both primary antibodies.

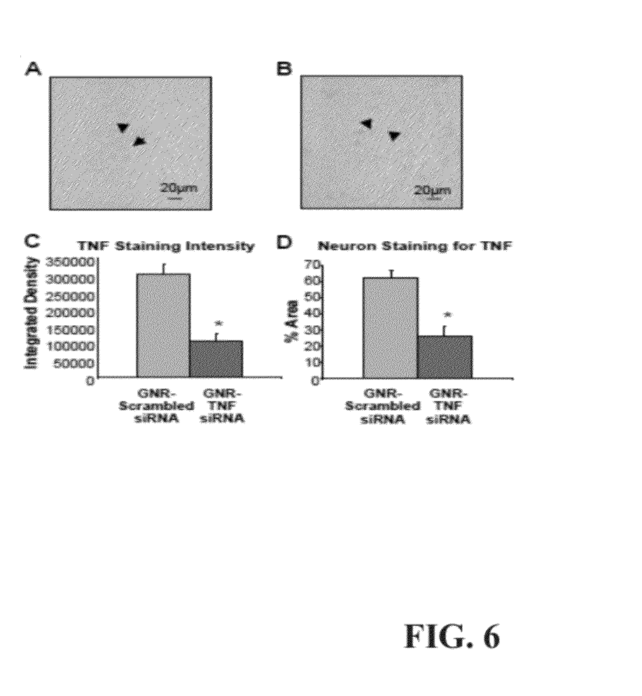

Turning now to FIG. 6, shown are quantified immunoreactive staining for TNF in the rat hippocampus. As demonstrated, TNF immunoreactivity is less in hippocampal sections from rats bilaterally microinjected with GNR-TNF-siRNÂCy3 into the CA1 region as compared to GNR-scrambled siRNA microinjected rats (* P<0.001, t-test). Therefore, it is shown that TNF gene expression specifically in the rat hippocampus can be suppressed. Shown in FIG. 6 is bilateral GNR-scrambled or TNF-siRNA (0.1 nmol/3 μl; 0.5 μl/min) micro-injection into the CA1 region of the rat hippocampus after 4 days. FIGS. 6 (A and B) show immunohistochemical staining (DAB) for TNF-α in acetone-fixed coronal hippocampal sections. In FIG. 6(A) are hippocampal sections prepared from rats microinjected with GNR-scrambled siRNÂCy3 (n=2). In FIG. 6(B) are hippocampal sections prepared from rats microinjected with GNR-TNFsiRNÂCy3 (n=2). Staining for TNF is evident in neurons in the characteristic hippocampal “jelly-roll” region (indicated by the black arrows in FIG. 6(A)) in sections prepared from rats microinjected with GNR-scrambled siRNÂCy3.

Staining for TNF is decreased or absent in neurons (indicated by the black arrows in FIG. 6(B)) from sections of rats microinjected with GNR-TNFsiRNÂCy3. FIGS. 6(C and D) demonstrate a quantitative analysis of TNF immunoreactive staining. FIG. 6(C) is a statistical analysis of integrated density values (t-test) in hippocampal sections prepared from rats bilaterally microinjected with 0.1 nmol/3 μl GNR-TNFsiRNA into the CA1 region as compared to the hippocampus from GNR-scrambled siRNA microinjected rats (*P<0.001). FIG. 6(D) is a statistical analysis of the % of the area of individual neurons (n=10/high filed (200 X) area/section) that stained for TNF in hippocampal neurons from GNR-TNFsiRNA microinjected rats as compared to GNR-scrambled siRNA injected rats (*P<0.001, t-test). As these preliminary studies consisted of n=2 for each group of rats, statistical analysis was performed on n=10 neurons/high field (200X) area in two areas from similar sections per group. Images were analyzed using ImageJ 1.32j software (NIH, USA, http://rsb.info.nih.gov/ij/) with the Color Deconvolution plug-in to perform stain separation (hematoxylin and DAB).

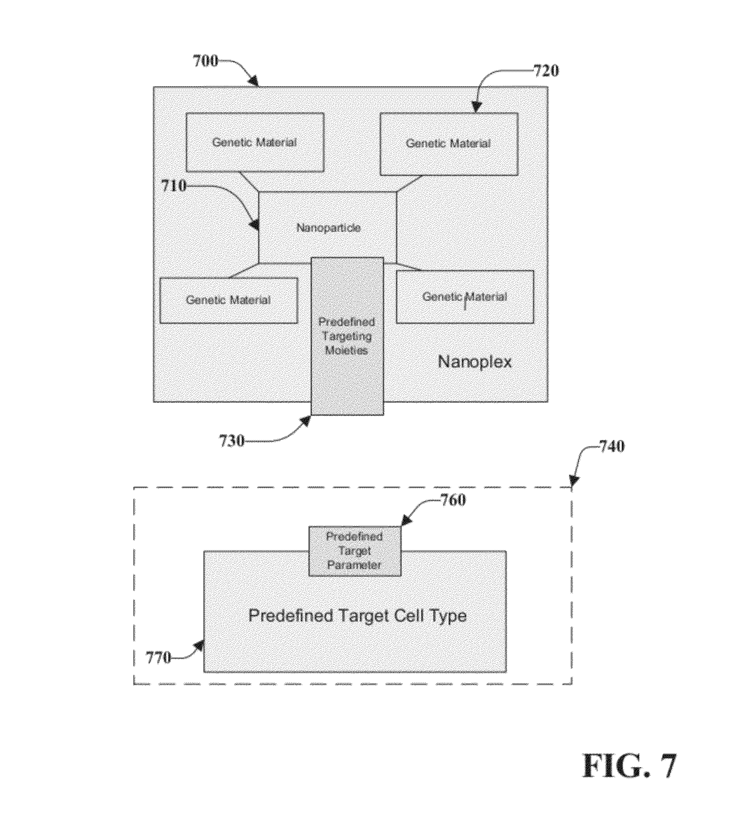

Turning now to FIG. 7, in another embodiment, product 700 comprises a nanoparticle 710 that can pair to genetic materials 720 that are N plasmids which enable the predefined target cell type 770 to up-regulate at least one of the anti-inflammatory cytokines IL-4, 1L-10, or IL-13, wherein N is an integer. The nanoparticle 710 can pair to genetic materials 720 that are siRNA and also genetic materials 720 that are cytokine antagonists (inhibit cytokine production) or cytokine agonists (enhance cytokine production) in some embodiments. Cytokines are regulators of host responses to infection, immune responses, inflammation, and trauma. Some cytokines when in-balanced act to make diseases worse (pro-inflammatory cytokine) whereas other cytokines reduce inflammation and promote healing (anti-inflammatory cytokine). Interleukins are a class of proteins produced by numerous cell-types, including, but not limited to, monocytes and some macrophages. These proteins have important physiological effects on a number of different cells involved in the inflammatory and immune responses of a subject. In an embodiment, genetic materials 720 can be plasmids (e.g. recombinant-DNA, cDNA clone, analogs, . . . ) which encode for and direct production of anti-inflammatory cytokines designed to block the action of pro-inflammatory cytokines and/or pro-inflammatory interleukins that act downstream to TNF-α production.

Furthermore, nanoparticle 910 can also pair to any one or more cytokine antagonists, proteins, and/or interleukin inhibitor that form a fusion protein (e.g. etanercept), a monoclonal antibody (e.g. infliximab), a binding protein (e.g. onercept), an antibody fragment (e.g. CDP 870) or other types of molecules which are potent, selective, and specific inhibitors of the action of pro-inflammatory cytokines. A cytokine antagonist can take several forms, such as monoclonal antibodies, a soluble receptor that bind to a cytokine and inactivate the cytokine, two soluble receptors fused together to an immunoglobulin molecule (e.g. fusion protein such as etanercept). For example, nanoparticle 710 can be paired to a TNF-α inhibiting siRNA and paired to any one or more plasmids that encode for the production of downstream anti-inflammatory cytokine's, including, but not limited to, IL-4, IL-10, or IL-13 in order to promote upregulation of those anti-inflammatory cytokines, in addition to TNF-α inhibiting siRNA in predefined target cell type 770.

Accordingly, nanoparticle 710 can also be paired to genetic materials 720 that encode for the production of inhibitors of pro-inflammatory cytokines (e.g. anti-inflammatory cytokines) that act downstream of TNF-α in the TNF-α pathway, including, but not limited to, IL-1 or IL-6. Nanoparticle 710 can additionally bind to one or more TNF-α inhibiting siRNA in addition to the anti-inflammatory cytokine plasmids in order to enhance the efficacy of controlling TNF production by affecting upstream and downstream cascades of TNF production in predefined target cell type 770. Predefined target parameter 760 can be any affinity parameter that corresponds to predefined targeting moieties 730. For example, in an embodiment, product 700 can target microglial cells by targeting specific antigens located on the surface membrane of the glial cell. Thus, product 700 can target an astrocyte glial cell that express glial fibrillary acidic proteins (GFAP), a predefined target parameter 760. In an aspect, other predefined target parameter 760 can be CD11b(OX-42) or coronin 1A to target microglia. In another aspect, target parameter 760 can be doublecortin (DCX) antigen to target neurons for hippocampal targeting. Furthermore, target parameter 760 can be NeuN to target mature neuron, or neurofilaments to target neurons in general, or neuron specific beta-tubulin to target neurons in general. In another aspect, target parameter 760 can be transferrin receptors to target endothelial cells lining the BBB.

Furthermore, a plasmid is genetic material that carries genes that, when expressed, allow cells to produce a specific interleukin of interest. The genetic materials 920 that is a plasmid requires preparation of a DNA sequence that directs a host cell to produce various interleukins that are anti-inflammatory in nature. The DNA sequence will be cloned into a vector or paired to a vector, in this case nanoparticle 710, capable of being transferred into and replicated in a host cell or predefined target cell type 770, wherein the nanoparticle 710 is also paired to operational elements needed to express the DNA sequence. Nanoparticle 710 can transfer the nanoparticle containing the DNA sequence and operational elements into the host cell or predefined target cell type 770 in order to express the plasmid DNA sequence that encodes for production of an anti-inflammatory cytokine, protein or cytokine inhibitor. The plasmid DNA sequence can include a synthetic DNA sequence, a natural DNA sequence, a cDNA sequence, genomic DNA segment, fragment of a natural DNA sequence.

Plasmids attached to nanoparticle 710 are also efficient cloning vectors. To be used in this way, the plasmid can contain at least one origin of replication, a multiple cloning site (called a polylinker) where a variety of restriction enzymes can cut so that foreign DNA can be inserted, a selectable genetic marker, and transcription and translation signals recognized by the host cell, so that the expression of a cloned gene can be easily identified. The plasmid DNA sequence can be inserted into nanoparticle 710 wherein such nanoparticle 710 delivers genetic materials 720 into a host cell or predefined target cell type 770 by nanoparticle 710 and allows for replication within the cell. A plasmid can possess restriction sites and/or contain operational elements required for transcription of the plasmid DNA sequence.

Plasmids can possess features such as: a minimal number of host-organism sequences, be maintained and propagated in a host cell or predefined target cell type 770, be present in a high copy number in the desired host cell or predefined target cell type 770, possess a regulatable promoter positioned for promoting regulation of the gene of interest, possess at least one marker DNA sequence coding for a trait present on a portion of the plasmid separated from that where the DNA sequence will be inserted, and/or possess a DNA sequence capable of terminating transcription. Furthermore, operable elements present on plasmids include, but are not limited to, any one or more of: a promoter, Shine-Dalgarno sequence, initiator codon, terminator codon, leader sequence for proteins, one gene for a regulator protein, other DNA sequences necessary or preferred for transcription, or DNA sequences necessary for translation. In another aspect, the plasmids genetic material can comprise a promoter, translational start signal, translational or a transcriptional stop signal. Furthermore, the plasmid can be controlled for a period of time

Furthermore, in an aspect, the nanoparticle 710 paired to the plasmid can traverse the blood brain barrier to target predefined cell type 770. In another aspect, the plasmid can contain an origin sequence, that is, a nucleotide sequence that codes for commencement of anti-inflammatory cytokines IL-4, IL-10, or IL-13 production. A lox site is a nucleotide sequence that is recognized by Cre recombinase enzyme. Pre recombinase enzyme is an enzyme which mediates the excision and integration of DNA based on specific lox sites through cleavage and ligation. A donor plasmid means a plasmid whereby the donor gene is flanked by lox sites on either side of the gene. An acceptor plasmid is a plasmid whereby a negative selected marker is flanked by lox sites. In an aspect, a nanoparticle 710 will be paired to a donor plasmid that contain an origin sequence (encoding for production of any one of: IL-4, IL-10, or IL-13) flanked with lox sites (e.g. wild-type lox site, incompatible lox site, half-mutant lox site, . . . ). After delivery of the donor plasmid to the predefined target cell type 770 by product 700, expression of the gene takes place, thus a respective anti-inflammatory cytokine will be produced. Additionally, nanoparticle 710 can be paired to an acceptor plasmid, for instance a plasmid encoding for production of Cre recombinase enzyme, wherein the acceptor plasmid can be delivered to a predefined target cell type 770 (Cre expressing cell), express the acceptor plasmid, thereby producing Cre recombinase. Upon expression of the Cre recombinase enzyme the lox sites will be activated (e.g. cleavage, mutation, recombination, ligation of plasmid can take place) leading to shutting down expression of IL-4, I1-10, or 11-13 in predefined target cell type 770.

In another embodiment, genetic materials 720 are Q cytokine inhibiting siRNA sequences that enables the predefined target cell type to downregulate at least one of TNF, IL-1 or IL-6, wherein Q is an integer. With respect to anti-inflammatory cytokines, the term up-regulate means an increased quantity of anti-inflammatory cytokines are released by target 770. Furthermore, with respect to pro-inflammatory cytokines (e.g. TNF, IL-1, IL-6, etc . . . ), the term downregulate means a decreased quantity of pro-inflammatory cytokines are released by target 770 770. Additionally, in another embodiment, the genetic materials 720 are any one or more of the L tumor necrosis factor (TNF) inhibiting siRNA sequences that respectively silences genes that produce tumor necrosis factor (TNF), the Q plasmids that upregulates at least one of IL-4, IL-10, or IL-13, or Q cytokine inhibiting siRNA sequences that downregulate at least one of IL-1 or IL-6, wherein L, N, and Q are integers. With respect to anti-inflammatory cytokines, the term upregulation means an increased quantity of anti-inflammatory cytokines are released by predetermined target cell type 770. Furthermore, with respect to pro-inflammatory cytokines, the term down-regulation means a decreased quantity of pro-inflammatory cytokines are produced or released by predetermined target cell type 770.

Accordingly, nanoparticle 710 can be paired to any combination of plasmids that code for upregulation of anti-inflammatory cytokine. For example, nanoparticle 710 can be paired to a plasmid that codes for increased expression of IL-4, and is paired to a TNF-a inhibiting siRNA simultaneously. In such an embodiment, product 700 has a greater likelihood of efficaciously reducing the symptoms associated with AD (e.g. Nft formation, β-amyloid plaque formation, . . . ). Furthermore, product 700 can target microenvironment 740, wherein the microenvironment is a brain region. The targeting feature in the brain region may provide the most therapeutic benefit for the subject. Additionally, other therapies are not able to treat neurological disorders due to an inability to enter the brain and cross the blood brain barrier (BBB), however, product 700 comprises nanoscale properties that allow product 700 to cross the BBB. Additionally, predefined targeting moieties 730 can include one moiety to take product 700 across the BBB and another moiety to target product 700 to a predefined target cell type 770.

In an embodiment, product 700 is used for treatment in AD by lessening, suppressing from progression or eliminating localized pathological changes induced by TNF productions. For instance product 700 can lessen, suppress from progression, eliminate, decrease or prevent an increase of at least one or more of β-amyloid formation, neurofibrillary tangle formation, senile plaque formation, neuritic plaque formation, or neuronal loss. Furthermore, in an embodiment, product 700 can lessen, suppress from progression, eliminate, decrease or prevent an increase of at least one or more of β-amyloid formation or neurofibrillary tangle formation by facilitating functioning of tau protein in brain cells. Tau protein is a protein that is normally found associated to the microtubules and functions in assembling and stabilizing the microtubules against the dynamic instability and linking of microtubules to filaments of the cytoskeleton. Tau proteins are abundant in neurons of the central nervous system, but are expressed at low levels in central nervous system astrocytes and oligodendrocytes. Neurofibrillary tangles are composed of tau, a microtubule-associated protein, which undergoes abnormal hyper-phosphorylation in AD. Increased phosphorylated tau leads to protein misfolding and aggregation resulting in the development of ‘tangles’. When tau proteins are defective and no longer stabilize microtubules properly, the subject can develop AD. Thus by silencing TNF production in the aging brain, hyper-phosphoylation of tau proteins will be prevented thereby facilitating the functioning of tau proteins in brain cells and cells of the central nervous system, and such AD symptoms such as β-amyloid formation, neurofibrillary tangle formation, senile plaque formation, neuritic plaque formation, or neuronal loss may be lessened.

In an embodiment, a therapeutically effective dosage level of product 700 can be administered to a subject. A therapeutically effective dosage level means an amount of product 700 in a medium, carrier, vehicle, or device suitable for administration to a subject. At therapeutically effective dosage level can take the form of any one or more of suspensions, emulsions, solutions, aerosols, powders, and the like. In another embodiment, nanoparticle 710, nanoparticle 210 or nanoparticle 410 can take the form of any one or more of a biodegradable polymer, tetrapod quantum dot, tetrapod article, multi-legged luminescent nanoparticle, tetrapod nanocrystal, biodegradable nanoparticle. Liposome, nanocarrier, dendrimer or other such nanoparticles and nanomaterials.

In various embodiments, the nanoparticle 710, nanoparticle 210 or nanoparticle 410 can consist of any nanoparticle, nanoparticulate or nanocrystal of any shape, size or form including but not limited to a polymer, lipid, dendrimer, dendrimer-type polymer, branch-type polymer, decomposable polymer, dendrimer-type structure, carbon nanotube, ceramic nanoparticle, nanosphere, metal nanoshell, quantum dot, nanorod, nanocrystal, liposome nanoparticle, iron oxide nanoparticle, polymeric nanoparticle, fullerene, liquid crystal, supermagnetic nanoparticle, colloid, nanopowder, nanocup, nanosphere, nanodiamond, nanostar, nanowire, plasmid and other nanoparticles, including those nanoparticles that possess a cationic or anionic charge.

In one embodiment, the nanoparticle 710, nanoparticle 210 or nanoparticle 410 can be a luminescent semiconductor nanocrystal compound comprised of a semiconductor nanocrystal capable of luminescence and/or absorption and/or scattering or diffraction when excited by an electromagnetic radiation source (of broad or narrow bandwidth) or a particle beam, and capable of exhibiting a detectable change in absorption and/or of emitting radiation in a narrow wavelength band and/or scattering or diffracting when excited. The semiconductor compound can be an element which includes but is not limited to Group II-IV semiconductor, Group III-V semiconductor, or MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, or HgTe.

In one embodiment, the nanoparticle 710, nanoparticle 210 or nanoparticle 410 can be a nanoparticle, wherein the nanoparticle is a material including but not limited to any noble metal, cadmium selenide, titanium, titanium dioxide, tin, tin oxide, silicon, silicon dioxide iron, iron̂III, oxide, silver, nickel, gold, copper, aluminum, steel, cobalt-chrome alloy, titanium alloy, brushite, tricalcium phosphate, alumina, silica, zirconia, diamond, polystyrene, silicone rubber, polycarbonate, polyurethanes, polypropylenes, polymethylmethaacrylate, polyvinyl chloride, polyesters, polyethers, or polyethylene.

In another embodiment, the nanoparticle 710, nanoparticle 210 or nanoparticle 410 can be a silver nanoparticle or silver composite. Besides silver, the silver composite may include either or both of (i) one or more other metals and (ii) one or more non-metals. Suitable other metals include, for example, Al, Au, Pt, Pd, Cu, Co, Cr, In, and Ni, particularly the transition metals, for example, Au, Pt, Pd, Cu, Cr, Ni, and mixtures thereof. Exemplary metal composites are Au—Ag, Ag—Cu, Au—Ag—Cu, and Au—Ag—Pd. Suitable non-metals in the metal composite include, for example, Si, C, and Ge. The various components of the silver composite may be present in an amount ranging for example from about 0.01% to about 99.9% by weight, particularly from about 10% to about 90% by weight. In embodiments, the silver composite is a metal alloy composed of silver and one, two or more other metals, with silver comprising, for example, at least about 20% of the nanoparticles by weight, particularly greater than about 50% of the nanoparticles by weight.

In yet another embodiment, the nanoparticle 710, nanoparticle 210 or nanoparticle 410 can be a nanoparticle that is a nanotube with a hollow tubular body defining an inner void, containing an open end on either side of the tube. In another embodiment, the nanoparticle 910 can be of any ceramic material wherein one metal alkoxide or metal salt can be selected from but is not limited to Al, Ba, Mg, Ca, La, Fe, Si, Ti, Zr, Pb, Sn, Zn, Cd, As, Ga, Sr, Bi, Ta, Se, Te, Hf, Mg, Ni, Mn, Co, S, Ge, Li, B and Ce to be used as the ceramic material of the ceramic nanparticle. In an embodiment, the nanoparticle 910 can be a nanometer-sized, hollow, spherically-shaped object that can be utilized to encapsulate small amounts of pharmaceuticals, enzymes, or other catalysts.

In one embodiment, the nanoparticle 710, nanoparticle 210 or nanoparticle 410 can be a polymer nanoparticle including but not limited to a synthetic polymers such as poly(ethylene glycol) (PEG), N-(2-hydroxylpropyl)methacrylamide (HPMA) co-polymers, poly(vinlylpyrrolidone), poly(ethyeneimine), and linear polyamidoamines; natural polymers such as dextran, dextrin, hyaluronic acid, collagen, and chitosans; pseudosynthetic polymers such as poly(L-lysine), poly(L-glutamic acid), poly(malic acid), and poly(aspartamides). Of these polymers, PEG, HPMA, dextran, and poly(L-lysine) have been used repeatedly in the development of nanoparticle carriers. The structural architecture of the polymer can be but is not limited to a spherical, linear, branched, cross-linked, block, graft, multivalent, dendronized, or star-shaped structure.

In one embodiment, the nanoparticle 710, nanoparticle 210 or nanoparticle 410 can be nanometer-scale composite structures composed of organic molecules intimately incorporated with inorganic molecules. In another embodiment, the nanoparticle 710, nanoparticle 210 or nanoparticle 410 can be a nanometer-scale wire made of materials that conduct electricity. They can be coated with molecules such as antibodies that will bind to proteins and other substances. In one embodiment, the nanoparticle 710 can be a biodegradable or non-biodegradable polymer defined by regular, highly branched monomers leading to a monodisperse, tree-like or generational structure with functional groups on the surface. The dendritic nanoparticle can vary by molecular weight and include, but not be limited to, dendronized polymers, hyperbranched polymers, a polymer brush. The dendrimer can be water soluble or non-water soluble. In one embodiment the nanoparticle component of the nanoplex can be a chitosan particle. A chitosan particle is a linear polysaccharide composed of randomly distributed β-(1-4)-linked D-glucosamine (deacetylated unit) and N-acetyl-D-glucosamine (acetylated unit).

In one embodiment, the nanoparticle 710, nanoparticle 210 or nanoparticle 410 can comprise of or pair to iron capable of serving theranostic function in relation to AD whereby the nanoparticle can deliver a genetic material payload while simultaneously allowing for MRI imaging of the nanoparticle delivery. Theranostic function means a material that can simultaneously have diagnostic, sensor and/or therapeutic functions.

The teachings of U.S. Provisional Pat. App. No. 61510403, U.S. Provisional Pat. App. No. 61515468, and U.S. Provisional Pat. App. No. 61538961 are herein incorporated by reference. These patents teach other nanoparticles and nanomaterials that can be nanoparticle 210, nanoparticle 410, or nanoparticle 710.

In an embodiment, nanoparticle 710, nanoparticle 210 or nanoparticle 410 can be a biodegradable polymer that control releases at least one or more TNF inhibiting siRNA sequence or genetic material. A biodegradable polymer is a molecule between 0.001 nm and 0.999 nm in size and dimensions, containing one or more copies of one or more constitutional units, commonly referred to as monomers, wherein the molecule can initiate, enhance or improve biocompatibility with the ability to degrade, sometimes at an adjustable rate of biodegradation. Monomers may refer to the free monomers and those that are incorporated into polymers. A biodegradable polymer may take on a number of configurations, which may be selected, for example from cyclic, linear, or branched configurations, among others. Branched configurations include star-shaped configurations (e.g. configurations in which three or more chains emanate from a single branch point), comb configurations (e.g. configurations having a main chain and a plurality of side chains, also referred to as “graft” configurations), dendritic configurations (e.g. arborescent and hyperbranched polymers), network configurations (e.g. crosslinked polymers) and so forth.

In another embodiment, product 700 comprises genetic materials 720 that at least inhibits TNF-α production or decreases TNF-α production. The product 700 aims to treat AD by decreasing TNF-α production or decreases TNF-α production through inhibition of one or more genes that, when expressed at the mRNA level, produce TNF-α and accordingly by delivering an siRNA to predefined target cell type 770 that produce TNF-α. Furthermore, in another aspect, product 700 comprises genetic materials 720 that inhibits mRNA from producing TNF-α. An siRNA suppresses the expression of a gene by targeting the mRNA of a gene having a complementary base sequence to the siRNA. In other words, an mRNA regulating the expression metabolic process of a specific gene is singularly degraded to stop the protein synthesis of the target gene, thereby treating a disease. In some aspects, nanoparticle 710, nanoparticle 210 or nanoparticle 410 can be a cationic liposome or micelle with a strong negative charge in order to deliver the siRNA into the body. In another aspect, the nanoparticle can be capable of neutralizing negative charges to allow in vivo permeation.

In another embodiment, nanoparticle 710, nanoparticle 210 or nanoparticle 410 can be further paired to at least one or more of TNF antagonist selected from the group consisting of etanercept soluble TNF receptor Type I, pegylated soluble TNF receptor TYPE 1 (PEGs TNF-R1), or onercept. TNF antagonists are proteins (e.g. recombinant, modified, . . . ) which bind to an neutralize TNF as a means to inhibit TNF activity. Etanercet, Infliximab, etanercept soluble TNF receptor Type I, pegylated soluble TNF receptor TYPE 1 (PEGs TNF-R1), and onercept are TNF antagonists authorized for use in treatment of rheumatoid arthritis. The TNF antagonists may also be used for treatment of Alzheimer's disease, Pick's disease. Lewy Body disease, or Idiopathic dementia. TNF antagonists include but are not limited to etanercept, infliximab, D2E7, a human anti-TNF monoclonal antibody, CDP 571, CDP 870, soluble TNF receptor Type I, pegylated soluble TNF receptor Type I (PEGs TNF-R1)), and onercept. All such TNF antaganosits can be paired to nanoparticle 710 nanoparticle 210 or nanoparticle 410 as genetic materials 720. In an aspect, antagonists of interleukin-1 such as recombinant IL1-RA, IL-1 receptor Type 2, and IL-1 Trap may be paired to nanoparticle 710, nanoparticle 210 or nanoparticle 410 as genetic materials 720, genetic materials 220, or genetic materials 420.

In an embodiment, the nanoparticle 710 is further paired to genetic materials 720 that at least cause one or more of increased IL-10 production, increased IL-4 production, decreased IL-1 production, decreased 1L-6 production, or increased IL-13 production. Interletikin 10 (IL-10) is an anti-inflammatory cytokine that regulates the inflammatory response by acting competitively against the activity of pro-inflammatory cytokines. Polymorphisms present in the promoter region of IL-10 are associated with the progression of AD. Up-regulation refers to the expression of a gene, or level of RNA or equivalent RNA encoding one or more protein subunits. For example, the expression of a protein, such as IL-4 or IL-10, can be increased in order to treat, prevent, ameliorate, or modulate a pathological condition (e.g., Alzheimer's disease, Pick's disease, Lewy Body disease, or Idiopathic dementia) caused or exacerbated by an absence or low level of gene expression, protein production, or cytokine production. Thus, nanoparticle 710 can be paired to genetic materials that can increase IL-10 and/or IL-4 production in a microenvironment 740 (e.g. the brain). Interleukin 4 (IL-4) is a pleiotropic cytokine and by increasing production of IL-4, a pathological condition (e.g., Alzheimer's disease, Pick's disease, Lewy Body disease, or Idiopathic dementia) can be treated, prevented, ameliorated, or modulated.

The term down-regulate refers to the expression of a gene, or level of RNA or equivalent RNA encoding one or more protein subunits, to inhibit the amount of protein production or cytokine production. Interleukin 1 (IL-1) is a pro-inflammatory cytokine (interleukin) that includes a family of cytokines involved in chronic inflammatory conditions such as Alzheimer's disease, Pick's disease, Lewy Body disease, or Idiopathic dementia. Interleukin 6 (IL-6) is an interleukin that acts as a pro-inflammatory cytokine and it mediates some anti-inflammatory effects by blocking further production of TNF and IL-1. Whereas IL-1 and TNF-α induce synthesis of each other, as well as IL-6, IL-6 terminates this upregulatory cascade and inhibits IL-1 and TNF-α synthesis. Interleukin 13 (IL-13) is a pleiotropic cytokine that inhibits production of pro-inflammatory cytokines IL-6, TNF-α. By decreasing or down-regulating the expression of a protein, such as IL-1, IL-6, and/or TNF, such decrease can treat, prevent, ameliorate, or modulate a pathological condition (e.g., Alzheimer's disease, Pick's disease, Lewy Body disease, or Idiopathic dementia) caused or exacerbated by a high level of gene expression, protein production, or cytokine production. For example, nanoparticle 710 can be paired to genetic materials 720 that are TNF inhibiting siRNA sequences and paired to genetic materials 720 that is an IL-1 decreasing gene. The efficaciousness of product 700 can be enhanced with such embodiments of the product.

Product 700, product 200 or product 400, and other non-limiting embodiments of the product (collectively referred to as “product embodiments”), in some embodiments, can be administered by any one or more of intrathecal injection or administration into a perispinal space. In another embodiment, product embodiments can be configured to be delivered through at least one or more of: aerosolized inhaler, intravenous, intra-articular, intra-thecal, peri-spinal, oral tablet, or topically. Product embodiments can take the form of a therapeutic composition, which contain various salts, buffers, pharmaceutical excipient (e.g. calcium carbonate, calcium phosphate, various diluents, various sugars, types of starch, cellulose derivatives, gelatin, vegetable oils, polyethylene glycols, . . . ) and/or hydrates. Therapeutic compositions of product embodiments can be administered to a subject in a manner that is pharmacologically useful.

The product embodiments can be part of a kit or device and can be filled into tubes, jars, bottles, aerosol containers, and any other form of packaging that will allow ease of application locally such as to the skin, rectum, vagina, mouth, hair, scalp, nose, and any other such superficial location. The product embodiments can also be made as a sterile dispersion and provided in a sealed tube or bottle for use on open wounds, fractures, burns, or infections. The product embodiments can be applied locally, either manually or by using a convenient applicator, for patient compliance and ease of applicability. The dose, number and frequency of applications can be decided by a person skilled in the art of treating local conditions such as a physician.

The product embodiments can also be used for delivery of active agents by the oral route. For delivery orally, the nanoparticle can be suspended in a palatable fluid such as a syrup or elixir, or incorporated into a solid dosage form. The nanoparticles can be filled into hard gelatin capsules either alone or in combination with suitable pharmaceutically acceptable excipients. The nanoparticles can also be converted into tablets and other solid dosage forms. The dispersion compositions of the invention can also be filled into soft gelatin capsules.

Suitable pharmaceutically acceptable excipients include diluents such as starch, lactose, dicalcium phosphate, tricalcium phosphate, microcrystalline cellulose, powdered cellulose, sucrose, mannitol, sorbitol, pregelatinized starch and the like or combinations thereof; binders such as acacia, guar gum, tragacanth, gelatin, starch, pregelatinized starch, polyvinyl pyrrolidone, hydroxypropyl cellulose, hydroxyethyl cellulose, hydroxypropyl methylcellulose and the like or combinations thereof; disintegrants such as starch, pregelatinized starch, sodium starch glycolate, croscarmellose sodium, polyvinyl pyrrolidone, crospovidone and the like or combinations thereof; lubricants such as stearic acid, magnesium stearate, zinc stearate and the like or combinations thereof; glidants such as colloidal silicon dioxide; anti-tacking agents such as talc; colorants; solubilizers such as anionic, cationic or zwitterionic and the like or combinations thereof; soft gelatin capsule shell components such gelatin, glycerin, propylene glycol, talc, colorants and water.

Therapeutic compositions of the several embodiments of the product can be provided as parenteral compositions or formulations, such as for injection or infusion. For example, parenteral formulations usually contain injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch or magnesium stearate. In addition, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The therapeutic compositions of the product embodiments can be prepared for use in prophylactic regimens (such as vaccines) and administered to any subject such as human or non-human subjects to elicit an response against AD. Thus, the pharmaceutical compositions typically contain a pharmaceutically effective amount of the product. Administration of therapeutic compositions of the several embodiments of the product can be by any common route as long as the target tissue (typically, the respiratory tract) is available via that route. This includes oral, nasal, ocular, buccal, or other mucosal (such as rectal or vaginal) or topical administration. Alternatively, administration will be by orthotopic, intradermal subcutaneous, intramuscular, intraperitoneal, or intravenous injection routes. Such therapeutic compositions are usually administered as pharmaceutically acceptable compositions that include physiologically acceptable carriers, buffers or other excipients. In the case of transdermal delivery routes, such transdermal administration include but not be limited to patch, gel, foam, sponge, cream, spray, ointment or combinations thereof.

In some product embodiments for administration of therapeutic compositions of the product, any inhaler device may be used including but not limited to pressurized metered does inhalers, breath-activated inhalers, inhalers with spacer devices, nebulisers. In some product embodiments for the transmucosal absorption administration, the administration may be accomplished by but is not limited to respiratory tract mucosal absorption, inhalation of vaporized, nebulized, powdered or aerosolized drug, as well as by direct instillation, oral transmucosal administration, sublingual administration, buccal administration, tablets, and nasal mucosal administration.

In various product embodiments, the therapeutic compositions of the product may be administered to the subject via any means including, but not limited to, gastrointestinal, enteral, central nervous system, epidural, intracerebral, intracerebroventricular, epicutaneous, intradermal, subcutaneous, nasal administration, intravenous, intraarterial, intramuscular, intracardiac, intraosseous infusion, intrasnovial, intrathecal, intraperitoneal, intravesical, intravitreal, intracavernous injection, intravaginal, intrauterine, transdermal, transmucosal, topical, epicutaneous, inhalational, enema, eye drops, ear drops, through mucous membranes, enteral, by mouth, by gastric feeding tube, by duodenal feeding tube, by gastronomy, rectally, pulmonary, buccal, ophthalmic, by bolus injection, via suppository drugs, intravenously, intra-arterial, intraosseous infusion, intra-muscular, inhalation, pill form, syrup, injection, by catheter, in dosage form, by drug injection, gas jet driven non-needle injection, intra-muscular needle injection, by hypodermic needle, by medical injection.

The therapeutic compositions of any product embodiments can also be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain about 100 mg of human serum albumin per milliliter of phosphate buffered saline. Other pharmaceutically acceptable carriers include aqueous solutions, non-toxic excipients, including salts, preservatives, buffers and the like may be used. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components of the pharmaceutical composition are adjusted according to well-known parameters.

Additional therapeutic compositions or formulations of any product embodiments are suitable for oral administration. Oral formulations can include excipients such as, pharmaceutical grades of mannitol, lactose, starch, magnesium stearate, sodium saccharine, cellulose, magnesium carbonate and the like. The compositions (medicaments) typically take the form of solutions, suspensions, aerosols or powders. In some embodiments, the therapeutic compositions of any of the embodiments of the product disclosed herein may be delivered via oral administration to a subject, and as such, these compositions may be formulated with an inert diluent or with an assimilable edible carrier, or they may be enclosed in hard- or soft-shell gelatin capsule, or they may be compressed into tablets, or they may be incorporated directly with the food of the diet.

The product may even be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration of the product embodiments of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as those containing sodium borate, glycerin and potassium bicarbonate, or dispersed in a dentifrice, including: gels, pastes, powders and slurries, or added in a therapeutically effective amount to a paste dentifrice that may include water, binders, abrasives, flavoring agents, foaming agents, and humectants, or alternatively fashioned into a tablet or solution form that may be placed under the tongue or otherwise dissolved in the mouth.. When the route is topical, the form may be a cream, ointment, salve or spray. Also, adhesive bandages could be used for the administration of vaccines.

In some product embodiments, the administration of the therapeutic compositions of the product may be by intranasal sprays, inhalation, and/or other aerosol delivery vehicles is also considered. Following formation, the product is made into a solution or suspension for aerosolization, using a pharmaceutically acceptable excipient. Suitable excipients will be those that neither cause irritation to the pulmonary tissues nor significantly disturb ciliary function. Excipients such as water, aqueous saline (with or without buffer), dextrose and water, or other known substances, can be employed with the subject invention. The exact concentration and volume of the solution are not critical, acceptable formulations being readily determined by those of ordinary skill in the art. The concentration and volume of the solution will generally be dictated by the particular nebulizer selected to deliver the complex, and, the intended dose. It is preferred to minimize the total volume, however, to prevent unduly long inhalation times for the subject.

In an aspect, the therapeutic compositions of any of the product embodiments may be, directly to the lungs, the product is aerosolized by any appropriate method. Usually, the aerosol will be generated by a medical nebulizer system which delivers the aerosol through a mouthpiece, facemask, etc. from which the subject can draw the aerosol into the lungs. Various nebulizers are known in the art and can be used in the method of the present invention. The selection of a nebulizer system will depend on whether alveolar or airway delivery (e.g., trachea, pharynx, bronchi, etc.), is desired. Examples of nebulizers useful for alveolar delivery include but are not limited to the Acorn 1 nebulizer, and the Respirgard II.®. Nebulizer System, both available commercially from Marquest Medical Products, Inc., Inglewood, Colo. Other commercially available nebulizers for use with the instant invention include the UltraVent.®. nebulizer available from Mallinckrodt, Inc. (Maryland Heights, Mo.); the Wright nebulizer (Wright, B. M., Lancet (1958) 3:24-25); and the DeVilbiss nebulizer (Mercer et al., Am. Ind. Hyg. Assoc. J. (1968) 29:66-78; T. T. Mercer, Chest (1981) 80:6(Sup) 813-817). Nebulizers useful for airway delivery include those typically used in the treatment of asthma. Such nebulizers are also commercially available. Likewise, transmucosal drug delivery in the form of a polytetrafluoroetheylene support matrix is a proper mode of administration of the product.

In some product embodiments, the therapeutic compositions of the product may be in the form of a sheet material. The device contains an acid-containing particulate polymeric resin dispersed throughout a polytetrafluoroethylene support matrix. There is a flexible film backing on one side of the device. The backing is preferably a flexible film that prevents bulk fluid flow and is inert to the ingredients of the device. The backing protects the composition from excessive swelling and loss of adhesion over the time period during which the composition is intended to remain adhered to the mucosal surface. In the case of a device that contains a drug intended to be delivered to or across a mucosal surface (as opposed to delivery to the vicinity of the mucosal surface, e.g., to the oral cavity), the film backing material is preferably substantially impermeable to the drug and therefore it effectively prevents migration of the drug out of the coated portion of the device. In the case of a device that contains a drug intended to be delivered, e.g., to the oral cavity or the vaginal cavity, the backing can be permeable to the agent to be delivered and can be permeable to saliva as well.

In some product embodiments, the therapeutic compositions of any of the embodiments of the product may be administered parenterally, intravenously, intramuscularly, or even intraperitoneally. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

In some product embodiments, the therapeutic compositions of any of the embodiments of the product can be an injectable particle that includes a substance to be delivered and a polymer that is bound to a biologically active molecule, wherein the particle is prepared in such a manner that the biologically active molecule is on the outside surface of the particle. Injectable particles with antibody or antibody fragments on their surfaces can be used to target specific cells or organs as desired for the selective dosing of drugs a wide range of biologically active materials or drugs can be incorporated into the polymer at the time of nanoparticle formation.

The substances to be incorporated should not chemically interact with the polymer during fabrication, or during the release process. Additives such as inorganic salts, BSA (bovine serum albumin), and inert organic compounds can be used to alter the profile of substance release, as known to those skilled in the art. Biologically-labile materials, for example, procaryotic or eucaryotic cells, such as bacteria, yeast, or mammalian cells, including human cells, or components thereof, such as cell walls, or conjugates of cellular can also be included in the particle. The term biologically active material refers to a peptide, protein, carbohydrate, nucleic acid, lipid, polysacccaride or combinations thereof, or synthetic inorganic or organic molecule, that causes a biological effect when administered in vivo to an animal, including but not limited to birds and mammals, including humans. Non-limiting examples are antigens, enzymes, hormones, receptors, and peptides. Examples of other molecules that can be incorporated include nucleosides, nucleotides, antisense, vitamins, minerals, and steroids.

The period of time of release, and kinetics of release, of the substance from the nanoparticle will vary depending on the copolymer or copolymer mixture or blend selected to fabricate the nanoparticle. Given the disclosure herein, those of ordinary skill in this art will be able to select the appropriate polymer or combination of polymers to achieve a desired effect.

In some product embodiments the therapeutic compositions of the product may be administered as a single or multiple daily subcutaneous injection. Several other methods delivery are now available or in development, including (a) continuous subcutaneous product infusion by a wearable infusion pump; (c) implantation of a programmable product pump; (d) oral, nasal, rectal and transdermal mechanisms of product delivery; (e) administration of product analogues; (0 implantation of polymeric capsules which give continuous or time-pulsed release of product.

Additionally, the product in any embodiment can be further comprised of at least one or more of: salt, ester, pharmaceutical excipient or hydrate. The therapeutic compositions of any of the embodiments of the product may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

The therapeutic compositions of any of the product embodiments can include any and all solvents, dispersion media, vehicles, coatings, diluents, antibacterial and antifungal agents, isotonic and absorption delaying agents, buffers, carrier solutions, suspensions, colloids, and the like. Supplementary active ingredients can also be incorporated into the compositions. In some aspects, esters, pharmaceutical excipients and/or hydrates can part of the therapeutic compositions of any of the embodiments of the product.

An effective amount of the therapeutic composition of the product embodiments is determined based on the intended goal, for example vaccination of a human or non-human subject. The appropriate dose will vary depending on the characteristics of the subject, for example, whether the subject is a human or nonhuman, the age, weight, and other health considerations pertaining to the condition or status of the subject, the mode, route of administration, and number of doses, and whether the pharmaceutical composition includes nucleic acids or viruses. Generally, the pharmaceutical compositions described herein are administered for the purpose of treating AD.

In particular examples, therapeutic compositions of product embodiments can include a disclosed therapeutic agent administered by sustained-release systems. Suitable examples of sustained release systems include suitable polymeric materials (such as, semi-permeable polymer matrices in the form of shaped articles, for example films, or microcapsules), suitable hydrophobic materials (for example as an emulsion in an acceptable oil) or ion exchange resins, and sparingly soluble derivatives (such as, for example, a sparingly soluble salt). Sustained release compositions can be administered orally, parenterally, intracistemally, intraperitoneally, topically (as by powders, ointments, gels, drops or transdermal patch), or as an oral or nasal spray. Sustained-release matrices include polylactides copolymers of L-glutamic acid and gamma-ethyl-L-glutamate.

In some product embodiments the therapeutic compositions the product can comprise polypeptides called receptor mediated permeabilizers (RMP) may be used, which, increase the permeability of the blood-brain barrier to molecules such as therapeutic agents or diagnostic agents. These receptor mediated permeabilizer A-7 or conformational analogues can be intravenously co-administered to a host together with molecules whose desired destination is the cerebrospinal fluid compartment of the brain. The permeabilizer A-7 or conformational analogues allow these molecules to penetrate the blood-brain barrier and arrive at this destination.

In an embodiment, the nanoparticle of any of the embodiments can comprise predefined targeting moieties that are one or more of an antibody or a protein. These predefined targeting moieties can assist in targeting the product to a specific predefined cell type depending on the characteristics and features associated with such predefined cell type.

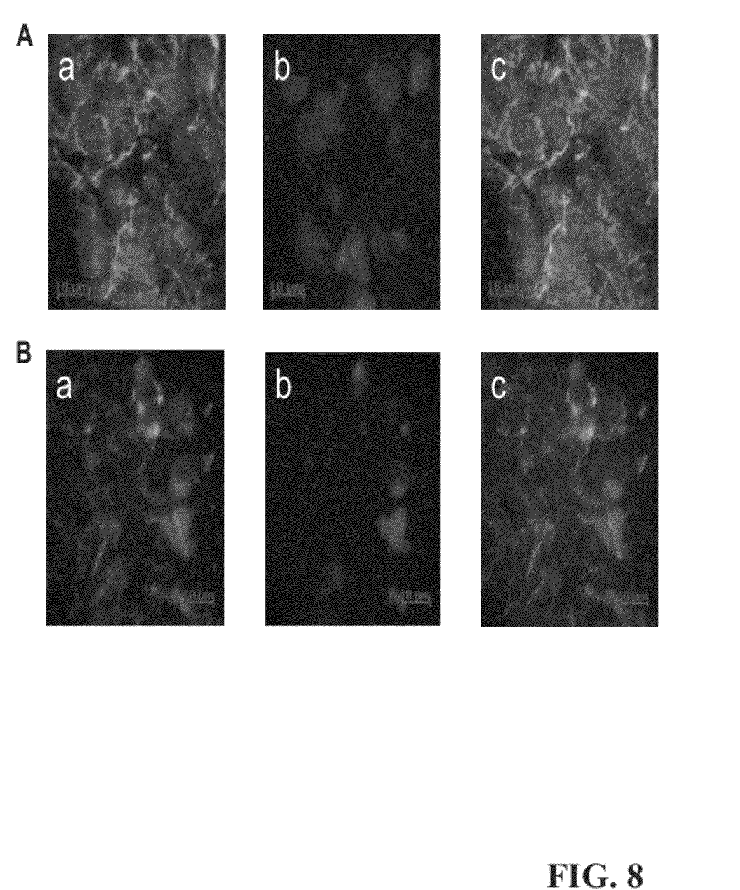

Turning now to FIG. 8, displayed are images that determine the cell type(s) in which the uptake of a nanoparticle paired to a plasmid (herein referred to as “nanoplasmidex-RFP” or “nanoplasmidex”) was associated in vivo, rat coronal hippocampal sections were labeled with primary antibodies to either glial fibrillary acidic protein (GFAP) (glial cells) or neurofilament-200 (NF-200) (neurons). Visualization of the primary antibody labeling was achieved using an IgGl-AlexaFluor 647 (AF-647) secondary antibody, with the excitation/emission maxima at 650/668 nm. The nanoplasmidex-RFP uptake into hippocampal cells was visualized in the same tissue sections, since the excitation/emission maxima of 555/584 nm for RFP provides emission in the orange region of the spectrum and is well separated from the far-red (AF-647) fluorophore emission, thereby facilitating a multicolor analysis. An overlay of the respective images (FIG. 8, Rows A and B, panel c) demonstrates that co-localization of nanoplasmidexes may occur with either neuronal (NF-200 positive; Row A) or glial (GFAP positive; Row B) cells (FIG. 8). Whereas the punctuate red staining together with the more diffuse RFP staining in Row A, panel b indicates production of TNF by neurons (NF-200 positive co-localization); the mainly diffuse RFP staining in Row B, panel b may indicate that the fusion protein has already been delivered to the glial cell (GFAP positive co-localization) plasma membrane, and the TNF released freeing the RFP to diffuse throughout the cytosol.

Turning now to FIG, 9, the bar graph illustrates the induction of gene expression of TNF 21 days after bilateral GNR-pTagrfp-Mutnf nanoplasmidex (150 ng/3 μl) microinjection into the rat hippocampal CA1 region. Data represents the mean ±S.E.M. of three separate experiments. Statistical significance indicated (*p<0.05) was by Student's t-test. CA1, left hippocampal CA1 region; CA3/DG, combined left hippocampal CA3/dentate gyrus regions; and PC, left parietal cortex. Results from qRT-PCR analysis of brain tissue harvested from rats receiving intra-hippocampal CA1injection of TNF nanoplasmidexes (150 ng/3 μl) display an increase in TNF mRNA expression (FIG. 8). Compared to values obtained after control nanoplasmidex injections, the TNF nanoplasmidexes induced an 85% increase in TNF mRNA expression in the CA1 region of the hippocampus.

Further disclosed, in an aspect is a nanoparticle that is a theranostic particle that allows for imaging in a subject and treatment of Alzheimer's Disease simultaneously. In an aspect, the theranostic particle can be iron oxide. Nanoparticles that that are Fe₂O₃, Fe₂O₂, Fe₃O₄ or other iron variations are magnetic materials that are biocompatible when used clinically and can serve as a diagnostic tool to view the location of the nanoparticles due to its ability to be observed by Magnetic Resonance Imaging (MRI). In another aspect, the product can comprise a theranostic nanoparticle known as a quantum dot wherein the nanoparticle can be imaged and deliver the genetic material pay load or other pay load to a predefined target cell type. The quantum dot can be any variety of shape, size and composition such as a tetrapod quantum dot or other quantum dots listed throughout this disclosure or incorporated by reference.

Further disclosed is a methodology, in accordance with certain aspects of this disclosure, for treating, Alzheimer's Disease, Pick's Disease, Lewy Body Disease, or Idiopathic Dementia comprising administering the dosage form of the product to a subject. In an aspect, the product administered to the subject is an amount effective to treat Alzheimer's Disease, Pick's Disease, Lewy Body Disease, or Idiopathic Dementia. In another aspect, the product is administered to a subject that has Alzheimer's Disease, Pick's Disease, Lewy Body Disease, or Idiopathic Dementia.

In accordance with certain aspects of this disclosure. While, for purposes of simplicity of explanation, the methodologies are shown and described as a series of acts, the disclosed subject matter is not limited by the order of acts, as some acts may occur in different orders and/or concurrently with other acts from that shown and described herein. For example, those skilled in the art will understand and appreciate that a methodology can alternatively be represented as a series of interrelated states or events, such as in a state diagram. Moreover, not all illustrated acts may be required to implement a methodology in accordance with the disclosed subject matter. Additionally, it is to be appreciated that the methodologies disclosed in this disclosure are capable of being composed as a pharmaceutical formulation to facilitate administering such products and/or methodologies to subjects.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described in this disclosure. Furthermore, not all illustrated acts may be required to implement the methodologies in accordance with certain aspects of this disclosure.

What is claimed is:

1. A product comprising:
a nanoparticle paired to W genetic materials that regulates inflammation in a microenvironment and X predefined targeting moieties that correspond to Y predefined target parameters associated with Z predefined target cell type m connection with treatment of at least one of the following Alzheimer's disease, Pick's disease, Lewy Body disease, or Idiopathic dementia, wherein W, X, Y, and Z are integers.

2. The product of claim wherein the genetic materials L tumor necrosis factor inhibiting small inhibitory RNA (siRNA,) sequences that respectively silences M genes that produce a tumor necrosis factor (TNF), wherein L and M are integers.

3. The product of claim 2, wherein the TNF is any one or more of TNF-α or TNF-β.

4. The product of claim 1, wherein the genetic materials are N plasmids that enables the predefined target cell type to upregulate at least one of IL-4, IL-10, or IL-13, wherein N is an integer.

5. The product of claim 1, wherein the genetic materials are Q cytokine inhibiting siRNA sequences that enables the predefined target cell type to downregulate at least one of IL-1 or IL-6, wherein Q is an integer.

6. The product of claim 1, wherein the genetic materials are any one or more of the L tumor necrosis factor (TNF) inhibiting siRNA sequences that respectively silences genes that produce tumor necrosis factor (TNF), the Q plasmids that upregulates at least one of IL-4, IL-10, or IL-13, or Q cytokine inhibiting siRNA sequences that downregulate at least one of IL-1 or 1L-6, wherein L, N, and Q are integers.

7. The product of claim 1, wherein the microenvironment is a brain region.

8. The product of claim 7, wherein the brain region is the hippocampus.

9. The product of claim 1, wherein the predefined cell type is any one or more of glial cells or neurons.

10. The product of claim 1, wherein the nanoplex composition decreases or prevents an increase of at least one or more of β-amyloid formation, and neurofibrillary tangle formation.

11. The product of claim 10, wherein the nanoplex composition decreases or prevents an increase of at least one or more of β-amyloid formation or neurofibrillary tangle formation by facilitating functioning of tau protein in brain cells.

12. The product of claim 1, wherein a therapeutically effective dosage level is administered to a subject.

13. The product of claim 1, wherein the nanoparticle is any one or more of a biodegradable polymer, tetrapod quantum dot, tetrapod article, multi-legged luminescent nanoparticle, tetrapod nanocrystal, biodegradable nanoparticle, liposome, nanocarrier, or dendrimer.

14. The product of claim 13, wherein the biodegradable polymer control releases at least one or more TNF inhibiting siRNA sequence or genetic material.

15. The product of claim 1, wherein the genetic materials at least inhibits TNF-α production or decreases TNF-α production.

16. The product of claim 1, wherein the genetic materials inhibits mRNA that produces INF-α.

17. The product of claim 1, wherein the nanoparticle is further paired to at least one or more of: TNF antagonist selected from the group consisting of entanercept soluble TNF receptor Type I, pegylated soluble TNF receptor TYPE I (PEGs TNF-R1), or onercept.

18. The product of claim 1, wherein the nanoparticle is further paired to a genetic materials that at least causes one or more of increased IL-10 production, increased IL-4 production, decreased IL-1 production, decreased IL-6 production, or increased IL-13 production.

19. The product of claim 1, wherein a therapeutically effective dosage is administered to a subject by any one or more of intrathecal injection or administration into a perispinal space.

20. The product of claim 1, wherein a therapeutically effective dosage is configured to be delivered through at least one or more of: aerosolized inhaler, intravenous, intra-articular, intra-thecal, peri-spinal, oral tablet, or topically.
